(19) United States
(12) Patent Application Publication
(10) Pub. No.: US 2008/0261261 A1
(43) Pub. Date: Oct. 23, 2008

(54) System/unit and method employing a plurality of magnetoelastic sensor elements for automatically quantifying parameters of whole blood and platelet-rich plasma

(75) Inventors: Craig A. Grimes, Boalsburg, PA (US); Kefeng Zeng, Mantua, NJ (US); Keat Ghee Ong, Houghton, MI (US); Xiping Yang, Dallas, TX (US)

Correspondence Address: JEAN M. MACHELEDT, 501 SKYSAIL LANE, SUITE B100, FORT COLLINS, CO, 80525-3133, US

(73) Assignee: KMG2 Sensors Corporation

(21) Appl. No.: 12/080,472

(22) Filed: Apr. 2, 2008

Related U.S. Application Data
(63) Continuation-in-part of application No. 11/710,294, filed on Feb. 23, 2007.
(60) Provisional application No. 60/787,945, filed on Mar. 31, 2006.
(60) Provisional application No. 61/007,495, filed on Dec. 12, 2007.

(51) Int. Cl. C12Q 1/02; C12M 1/00

(52) U.S. Cl. 435/29; 435/287.1

(57) ABSTRACT

A system/analyzer-unit and method/platform—using information obtained from at least one, adapted for a plurality of, magnetoelastic sensor elements in contact with one or more samples comprising blood from a patient—for automatically quantifying one or more parameters of the patient's blood. Information obtained from emissions measured from each of the sensor elements is uniquely processed to determine a quantification about the patient's blood, such as, quantifying platelet aggregation to determine platelet contribution toward clot formation; quantifying fibrin network contribution toward clot formation; quantifying platelet-fibrin clot interactions; quantifying kinetics of thrombin clot generation; quantifying platelet-fibrin clot strength; and so on. Structural aspects of the analyzer-unit include: a cartridge having at least one bay within which a sensor element is positioned; each bay in fluid communication with both (a) an entry port for injecting a first blood sample composed of blood taken from the patient (human or other mammal), and (b) a gas vent through which air displaced by injecting the first blood sample into the bay.

### PRIORITY BENEFIT TO CO-PENDING PATENT APPLICATIONS

This application claims the benefit of: (1) pending U.S. provisional Pat. App. No. 61/007,495 filed 12 Dec. 2007 describing developments of one of the applicants hereof, on behalf of the assignee; and (2) is a continuation-in-part (CIP) of pending U.S. patent application Ser. No. 11/710,294 filed 23 Feb. 2007 for the applicants on behalf of the assignee. The specification and drawings of both provisional app. No. 61/007,495 and the parent application Ser. No. 11/710,294 are hereby incorporated herein by reference, in their entirety, providing further edification of the advancements set forth herein.

### BACKGROUND OF THE INVENTION

### Field of the Invention

In general, the invention relates to systems and techniques for analyzing and characterizing mammalian blood clots, especially techniques that quantify and track parameters and properties thereof. Herein, focus is on a new system/analyzer-unit and method/platform—using information obtained from a plurality of magnetoelastic sensor elements in contact with one or more samples comprising blood from a patient—for automatically quantifying one or more parameters of the patient's blood. The new analyzer-unit and associated technique provides trained clinicians, surgeons, emergency room personnel, medical technicians—indeed, a wide variety of both human medical and veterinary care-providers—in the field, in the lab, in an operating room, and so on, with a handy, portable, non-invasive diagnostic tool for on-the-spot testing, periodic or long-term monitoring, to gather information about the condition of a patient's blood, whether of a critical nature or not.

Information obtained from emissions measured from each of the magnetoelastic sensor elements is uniquely processed to determine a quantification about the blood taken from a patient, such as, quantifying platelet aggregation to determine platelet contribution toward clot formation; quantifying fibrin network contribution toward clot formation; quantifying platelet-fibrin clot interactions; quantifying kinetics of thrombin clot generation; quantifying platelet-fibrin clot strength; and so on. The unique structure of the analyzer-unit permits simultaneous measurement to be made of emissions from several different sensor elements, of special interest in the event more than one quantitative assessment is sought of the patient's blood during a test.

so as to provide the information needed for processing to quantify/assess more than one blood parameter/property, automatically. The new analyzer-unit and method contemplated herein, allow assessments to be made about ‘whole blood’ or platelet-rich plasma (PRP) of a patient (any mammal, including humans and non-human mammals such as livestock, wildlife, and domesticated pets).

More-particularly, a first aspect of the invention is directed to a system/analyzer-unit and associated method for measuring emissions from a first, second, and third magnetoelastic sensor element while being exposed to a time-varying magnetic field. The method includes the steps of: measuring first emissions collected from a first magnetoelastic sensor element in contact with a first blood sample from a mammal; measuring second emissions collected from a second magnetoelastic sensor element in contact with a second blood sample from the mammal; measuring third emissions collected from a third magnetoelastic sensor element in contact with a third blood sample from the mammal; and processing information from the first, second, and third emissions so collected to make at least one quantitative assessment/quantification about the blood.

A second aspect of the invention is directed to a system/analyzer-unit and associated method for measuring emissions from a first and second magnetoelastic sensor element in contact with a first blood sample from a mammal while each of the elements is being exposed to a time-varying magnetic field. The emissions measured from the first magnetoelastic sensor element to provide first information relating to a property of the blood; the emissions measured from the second magnetoelastic sensor element to provide second information relating to a property of the blood, said first information being different from said second information, such that at least one quantitative assessment/quantification is made of/about the blood.

Excitation of resonator-type sensing elements. In earlier patented work, one of which is entitled “Magnetoelastic Sensor for Characterizing Properties of Thin-film/Coatings” U.S. Pat. No. 6,688,162, one or more of the applicants hereof detail the excitation of magnetoelastic elements, in operation as sensing units:
When a sample of magnetoelastic material is exposed to an alternating magnetic field, it starts to vibrate. This external time-varying magnetic field can be a time-harmonic signal or a non-uniform field pulse (or several such pulses transmitted randomly or periodically). If furthermore a steady DC magnetic field is superimposed to the comparatively small AC magnetic field, these vibrations occur in a harmonic fashion, leading to the excitation of harmonic acoustic waves inside the sample. The mechanical oscillations cause a magnetic flux change in the material due to the inverse magnetoelastic effect. These flux changes, in unison with the mechanical vibrations, can be detected in a set of EM emission pick-up coils. The vibrations of the sample are largest if the frequency of the exciting field coincides with the characteristic acoustic resonant frequency of the sample. Thus, the magnetoelastic resonance frequency detectable by an EM pick-up coil coincides with the frequency of the acoustic resonance. And, sensor element emissions can be detected acoustically, for example by a remote microphone/hydrophone or a piezoelectric crystal, by detecting the acoustic wave generated from the mechanical vibrations of the sensor. A relative-maximum response of the emissions remotely measured is identified to determine the sensing element's characteristic resonant frequency. The emissions from a sensing element of the invention can also be monitored optically whereby amplitude modulation of a laser beam reflected from the sensor surface is detected. Signal processing of the sensor elements can take place in the frequency-domain or in the time-domain using a field-pulse excitation.
. . .
FIG. 1A schematically depicts components of an apparatus and method of the invention for remote query of a thin-film layer or coating 14 atop a base magnetostrictive element 12. A time-varying magnetic field 17 is applied to sensor element 10, with a layer/coating 14 of interest having been deposited onto a surface of the base 14, by way of a suitable drive coil 16 such that emissions 19 from the sensor element can be picked-up by a suitable pick-up coil 18. Two useful ways to measure the frequency spectrum include: frequency domain measurement and the time domain measurement. In the frequency domain measurement, the sensing element's vibration is excited by an alternating magnetic field of a monochromatic frequency. The amplitude of the sensor response is then registered while sweeping (‘listening’) over a range of frequencies that includes the resonance frequency of the sensor element. Finding the maximum amplitude of the sensor response leads to the characteristic resonant frequency. FIG. 1B graphically depicts interrogation field transmissions from a drive coil (SEND) in both the frequency domain 22 and in the time-domain 26 (an impulse of, say, 200 A/m and 8 μs in duration). The transient response (emissions) captured 27 is converted to frequency domain 28 using a FFT to identify a resonant frequency. [end quote]

Applications/uses of resonator-type sensing elements. Tracking the resonant behavior of magnetoelastic resonator sensors has enabled physical property measurements including pressure, temperature, liquid density and viscosity, and fluid flow velocity and direction. Magnetoelastic sensors have been developed for the detection and quantification of a number of physical properties including pressure, temperature, liquid density and viscosity, flow velocity, and determining the elastic modulus of thin films. In combination with chemically active mass-elasticity changing films magnetoelastic chemical sensors have been used for gas-phase sensing of humidity, carbon dioxide, and ammonia. In combination with chemically active mass-elasticity changing films magnetoelastic chemical sensors have been used for liquid-phase sensing of pH, salt concentrations, glucose, trypsin, and acid phosphatase. Sensors for the detection of different biological agents including ricin, staphylococcal endotoxin B, and E. coli 0157:H7 have been fabricated by antigen-antibody coatings on the magnetoelastic sensor surface. Many of these prior systems were developed by the applicant hereof, as principal or a co-principal investigator.

U.S. Pat. No. 6,688,162, granted to L. Bachas, G. Barrett, *C. A. Grimes, D. Kouzoudis, S. Schmidt on 10 Feb. 2004, entitled Magnetoelastic Sensor for Characterizing Properties of Thin-Film/Coatings, “Bachas, et al. (2004),” provides basic technological background discussion concerning the operation of resonator-type sensor elements in connection with direct quantitative measurement of parameters and characteristics of an analyte of interest (in that case, especially one in the form of a thin film/layer atop a surface of the element). U.S. Pat. No. 6,688,162 to Bachas, et al. (2004) is incorporated herein by reference for its detailed background technical discussion of a sensing innovation co-designed by the applicant hereof, while obligated under an assignment to another assignee.

Another patent, U.S. Pat. No. 7,113,876, was granted for the threshold-crossing counting technique to three co-applicants hereof (Drs. K. Zeng, K. G. Ong, and C. A. Grimes). Other patents and published manuscripts that share at least one applicant hereof describe applications of resonator-type sensing elements in sensing an environment, itself, and/or the presence, concentration, chemical make up, and so on, of an analyte of interest (e.g., toxins or other undesirable chemical or substance, etc.), include: U.S. Pat. No. 6,639,402 issued 28 Oct. 2003 to Grimes et al. entitled “Temperature, Stress, and Corrosive Sensing Apparatus Utilizing Harmonic Response of Magnetically Soft Sensor Element(s);” U.S. Pat. No. 6,393,921 B1 issued 28 May 2002 to Grimes et al. entitled “Magnetoelastic Sensing Apparatus and Method for Remote Pressure Query of an Environment;” U.S. Pat. No. 6,397,661 B1 issued 4 Jun. 2002 to Grimes et al. entitled “Remote Magneto-elastic Analyte, Viscosity and Temperature Sensing Apparatus and Associated Method of Sensing;” Grimes, C. A., K. G. Ong, et al. “Magnetoelastic sensors for remote query environmental monitoring,” Journal of Smart Materials and Structures, vol. 8 (1999) 639-646; K. Zeng, K. G. Ong, C. Mungle, and C. A. Grimes, Rev. Sci. Instruments Vol. 73, 4375-4380 (December 2002) (wherein a unique frequency counting technique was reported to determine resonance frequency of a sensor by counting, after termination of the excitation signal, the zero-crossings of the transitory ring-down oscillation, damping was not addressed); and Jain, M. K., C. A. Grimes, “A Wireless Magnetoelastic Micro-Sensor Array for Simultaneous Measurement of Temperature and Pressure,” IEEE Transactions on Magnetics, vol. 37, No. 4, pp. 2022-2024, 2001.

Reference may be made, herein by way of example, to sensing and analysis samples of bovine blood (i.e., relating or belonging to the genus: Bos of ruminant animals that includes mammals often simply referred to as ‘livestock’, namely, cattle, oxen, and buffalo). The unique sensing element, associated sensing platform/device, and method contemplated hereby are intended and adapted for use in the analysis, diagnosis, and study of whole blood and platelet-rich plasma (PRP) of all mammals (occasionally, “mammalian blood” and “mammalian PRP”, or more-simply as, blood and PRP). Here, focus is on the use of magnetoelastic sensor elements to study platelet aggregation in whole blood or PRP, and for use in distinguishing fibrin and thrombin generated clotting cascades in whole blood or PRP.

### Further Historical Perspective: General Discussion by Way of Reference, Only

Blood clotting commonly represents a process of blood solidification that occurs upon external injury to tissue or blood vessels. Blood clotting is an essential part of the complex physiological process referred to as the coagulation cascade, or hemostasis, that requires a delicate balance between blood cells, platelets, coagulation and tissue factors. An injury to a blood vessel results in a series of enzymatic reactions between these various components with a final objective of stopping blood flow (clotting) at the wound site. While, in the case of an external injury it is desirable to form a clot in a short period of time to minimize blood loss, inside the body formation of even the smallest of clots can lead to a fatal hemorrhage. Conventional techniques for characterizing and analyzing blood clots are identified in The Clotting Times, October 2004, labeled ATTACHMENT A, hereof—the whole of which is incorporated herein by reference as a general technical background reference—page 6 discusses current techniques employed in the study of platelet function.

Platelets play a crucial role in the hemostasis process. Created in the bone marrow platelets have a half-life of 8-12 days in blood, during which they remain functional. The clotting cascade critically depends on the activation and aggregation of functional platelets, in particular for smaller blood vessels, where a vascular hole at the site of injury is blocked by a ‘platelet plug’ rather than by a blood clot. Standard platelet counts are 150,000/μL to 400,000/μL, while platelet counts lower than 50,000/μL often lead to spontaneous bleeding from capillary vessels, i.e. thrombocytopenia. Abnormal platelet count and activity influence other hemostatic disorders such as cerebrovascular disease, peripheral vascular disease and venous thromboembolism. An assessment of the platelet function, measured in terms of either platelet number or extent of aggregation, can be of critical importance for patients with hemostatic disorders.

Platelet aggregometry was first developed by Born in 1962 for platelet rich plasma (PRP); light transmission through the plasma was measured as a function of time after it was activated with adenosine di-phosphate (ADP) agonist. Previous to Born it had been shown that ADP caused platelets to form aggregates. Born showed that as the platelets formed aggregates under the influence of ADP the optical density of the plasma decreased, resulting in increased transmittance. The transparency of the plasma was directly proportional to the extent of aggregation which, in turn, was proportional to the number of functional platelets in the plasma. This technique has long been considered a standard in platelet aggregation studies. However, there are a number of issues that limit the utility of light transmission aggregometry.

Another technique, whole blood impedance aggregometry, requires an anti-coagulated whole blood sample to be diluted 1:1 with 0.9% saline, with two electrodes inserted into the blood to measure electrical impedance with time. As the platelets aggregate under the influence of an agonist such as ADP they adhere to the immersed electrodes resulting in a change of electrical impedance. The impedance change is proportional to the extent of platelet aggregation in the blood sample. Impedance aggregometry, although in use to study the platelet function of whole blood, likewise has limitations: It is insensitive to microaggregate formation.

Two other conventional methods for platelet aggregation studies of whole blood are: single platelet counting techniques and flow cytometry. The single platelet counting technique measures the fall in the number of platelets in a whole blood sample subjected to an agonist, with the reduction in the platelet number being proportional to the platelet aggregation. A modification of this technique has resulted in ‘flow cytometry’, which detects platelet aggregation in an ADP mixed blood sample labeled with platelet specific fluorescent antibodies. While flow cytometry may be able to detect both macro and micro-aggregate formation of platelets (since the fluorescent signal differs according to the size of the platelet clusters), the mixing of florescent markers leaves the blood sample open to contamination.

“A Modified Thromboelastographic Method for Monitoring c7E3 Fab in Heparinized Patients,” by Philip E. Greilich, MD, et al. Anesth Analg (1997) 84:31-8 (hereafter, Greilich, et al. 1997), describes an assay it refers to as “MTEG” for monitoring effects of potent antiplatelet drugs, stating:
The monoclonal antibody, c7E3 Fab, binds to the platelet surface fibrinogen receptor (GPIIb/IIIa), inhibiting platelet aggregation and clot retraction. [Thus, it is a potent antiplatelet drug.] We performed an in vitro study to assess the ability of a modification of the thromboelastograph (MTEG) to detect inhibition of clot strength by c7E3 Fab and its reversal with plateletrich plasma (PRP). In the modified assay (MTEG), thrombin was added to whole blood (WB) and platelet poor plasma (PPP) and the resultant maximum amplitude (MA) was measured, MAWB and MAppp, respectively.

“Use of abciximab-Modified Thrombelastography in Patients Undergoing Cardiac Surgery,” by S. C. Kettner, MD, et al. Anesth Analg (1999) 89:580-4 (hereafter, Kettner, et al. 1999), describes an assay it refers to as abciximab-modified Thrombelastography (TEG) for monitoring coagulation when abciximab-fab, a platelet function inhibitor, is used, as follows:
The maximum amplitude (MA) of TEG measures clot strength, which is dependent on both fibrinogen level and platelet function. Inhibition of platelet function with abciximab-fab is suggested to permit quantitative assessment of the contribution of fibrinogen to clot strength. We hypothesized that abciximab-modified TEG permits prediction of plasma fibrinogen levels and that the difference of standard MA and abciximab-modified MA (AMA) is a correlate for platelet function [p. 580] . . . .
. . . The use of standard TEG to distinguish between hypofibrinogenemia or platelet dysfunction as the cause of hypocoagulation is therefore ambiguous, because a decrease in MA can indicate either decreased plasma fibrinogen levels or reduced overall platelet function. Inhibition of platelet function allows quantitative assessment of the contribution of fibrinogen to clot strength . . . abciximab-fab is an antibody fragment that binds to platelet glycoprotein IIb/IIIa and blocks the interaction of platelets with fibrin in TEG . . . . Our data show that the blockade of platelet function by abciximab-fab antibody fragments enables prediction of fibrinogen levels, and ΔGMA correlates with platelet number. ΔGMA and abciximab MA can therefore help to distinguish between fibrinogen deficiency and platelet dysfunction and could guide transfusion of cryoprecipitate and platelets. Although ΔGMA correlates with platelet count in our study, we have not investigated whether ΔGMA correlates with other platelet tests or surgical blood loss. [p. 583]

### General Background Definitions, for Reference Only:

I. Mammalian Blood, Coagulation Cascade, etc.

Mammalian Blood is a biological fluid that circulates throughout mammals and consists of plasma and blood cells, namely, red blood cells (also called RBCs or erythrocytes), white blood cells (includes both leukocytes and lymphocytes), and platelets (also called thrombocytes). Blood plasma, the liquid component of blood in which blood cells are suspended, is predominantly water. However, it also contains many vital proteins including fibrinogen (a clotting factor), globulins and human serum albumin. Red blood cells are the most abundant cells in blood: They contain hemoglobin, an iron-containing protein, which facilitates transportation of oxygen and carbon dioxide. White blood cells help to resist infections. Platelets are important in the clotting of blood (as further explained).

Platelets, or thrombocytes, are the cells circulating in the blood that are involved in the cellular mechanisms of primary hemostasis leading to the formation of blood clots. Dysfunction or low levels of platelets predisposes a mammal to bleeding, while high levels may increase the risk of thrombosis. Platelet functions are generalized into several categories: adhesion and aggregation; clot retraction; pro-coagulation; cytokine signalling; and phagocytosis. Adhesion and aggregation refers to the activity of platelets to adhere to each other via adhesion receptors, or integrins, and to the endothelial cells in the wall of the blood vessel forming a haemostatic plug (or, clot) in conjunction with fibrin.

Coagulation is the complex process by which blood forms solid clots. It is an important part of hemostasis (the cessation of blood loss from a damaged vessel) whereby a damaged blood vessel wall is covered by a platelet- and fibrin-containing clot to stop bleeding and begin repair of the damaged vessel. Coagulation is initiated once an injury to a blood vessel lining occurs. Platelets immediately form a hemostatic plug at the site of injury; this is called primary hemostasis. Secondary hemostasis—which occurs simultaneously—is where proteins (coagulation factors) in the blood plasma respond in a coagulation cascade to form fibrin strands which strengthen the platelet plug. Disorders of coagulation can lead to an increased risk of bleeding, or clotting and embolism. Thrombosis is the pathological development of blood clots: an embolism is said to occur when a blood clot (thrombus) migrates to another part of the body.

Quantification is the act of quantifying, that is, of giving a numerical value to a measurement of something.

II. Blood Clotting Kinetics: Shown in FIG. 1A is the coagulation cascade: It has two pathways 10—or series of chemical reactions—that result in the formation of fibrin (12), the building block of a hemostatic plug (or, clot). The two pathways 10 that lead to fibrin formation are labeled by way of background reference as Contact Activation pathway and Tissue Factor pathway (also known as intrinsic and extrinsic pathways 10). The blood clotting process is recognized to occur in three stages, vascular spasm, platelet plug formation, and finally, blood clotting. In the first stage, prothrombinase is formed by the interaction of the different clotting factors that include calcium ions, enzymes, platelets and damaged tissues. Prothrombinase can be formed by either intrinsic or extrinsic pathways 10: the intrinsic pathway is initiated by liquid blood making contact with a foreign surface inside the blood vessel, whereas the extrinsic pathway occurs when the liquid blood comes in contact with an injured tissue. In the second stage of the clotting process prothombinase converts the protein prothombin into an enzyme thrombin. In the final stage, thrombin interacts with fibrinogen (a plasma protein synthesized in the body) into fibrin, which is insoluble and forms the polymer threads that binds the blood into a solidified mass.

III. Digital computers. A processor is the set of logic devices/circuitry that responds to and processes instructions to drive a computerized device. The central processing unit (CPU) is considered the computing unit of a digital electrically-driven or other type of computerized system. A conventional CPU, often referred to simply as a processor, is made up of a control unit, program sequencer, and an arithmetic logic unit (or, ALU)—circuitry that handles calculating and comparing tasks of a CPU. Numbers are transferred from memory into the ALU for calculation, and the results are sent back into memory. Alphanumeric data is sent from memory into the ALU for comparing. The CPUs of a computer may be contained on a single ‘chip’, often referred to as microprocessors because of their tiny physical size. As is well known, the basic elements of a simple computer include a CPU, clock and main memory; whereas a complete computer system requires the addition of control units, an operating system, and input, output and storage devices. The very tiny devices referred to as ‘microprocessors’ typically contain the processing components of a CPU as integrated circuitry, along with associated bus interface. A microcontroller typically incorporates one or more microprocessor, memory, and I/O circuits as an integrated circuit (IC). Computer instruction(s) are used to trigger computations carried out by the CPU.

IV. Computer Memory and Computer Readable Storage/Media. While the word ‘memory’ has historically referred to that which is stored temporarily, with storage traditionally used to refer to a semi-permanent or permanent holding place for digital data—such as that entered by a user for holding long term—more-recently, the definitions of these terms have blurred. A non-exhaustive listing of well known computer readable storage device technologies are categorized here for reference: (1) magnetic tape technologies; (2) magnetic disk technologies include floppy disk/diskettes, fixed hard disks (often in desktops, laptops, workstations, etc.), (3) solid-state disk (SSD) technology including DRAM and ‘flash memory’; and (4) optical disk technology, including magneto-optical disks, PD, CD-ROM, CD-R, CD-RW, DVD-ROM, DVD-R, DVD-RAM, WORM, OROM, holographic, solid state optical disk technology, and so on.

### SUMMARY OF THE INVENTION

Briefly described, in one characterization, the invention is directed to a system/analyzer-unit and associated method for measuring emissions from a first, second, and third magnetoelastic sensor element while being exposed to a time-varying magnetic field. The method includes the steps of: measuring first emissions collected from a first magnetoelastic sensor element in contact with a first blood sample from a mammal; measuring second emissions collected from a second magnetoelastic sensor element in contact with a second blood sample from the mammal; measuring third emissions collected from a third magnetoelastic sensor element in contact with a third blood sample from the mammal; and processing information from the first, second, and third emissions so collected to make at least one quantitative assessment/quantification about the blood.

In a second characterization, the invention is a system/analyzer-unit and associated method for measuring emissions from at least a first and second magneto-elastic sensor element in contact with a first blood sample from a mammal while each of the elements is being exposed to a time-varying magnetic field. The emissions measured from the first magnetoelastic sensor element to provide first information relating to a property of the blood; the emissions measured from the second magnetoelastic sensor element to provide second information relating to a property of the blood, said first information being different from said second information, such that at least one quantitative assessment/quantification is made of/about the blood.

As mentioned, information obtained from the emissions measured from each sensor element is uniquely processed to determine a quantification about the blood taken from a patient, such as, quantifying platelet aggregation to determine platelet contribution toward clot formation; quantifying fibrin network contribution toward clot formation; quantifying platelet-fibrin clot interactions; quantifying kinetics of thrombin clot generation; quantifying platelet-fibrin clot strength; and so on. In the event more than one quantitative assessment is sought of a patient's blood during a test, the unique structure of the analyzer-unit can make substantially-simultaneous measurements of emissions, to provide requisite information for automatic determination of a quantification of more than one blood parameter/property.

The new system/analyzer-unit and method using magnetoelastic sensor elements as contemplated herein, may also be employed for quantitative assessment of the blood of a patient to which some drug is being administered, for example, an antiplatelet drug (as typically administered, inhibit platelet aggregation and clot retraction).

Unique structural aspects of a new analyzer-unit include: a cartridge having at least one bay within which a magnetoelastic sensor element is positioned; each bay is in fluid communication with both (a) an entry port for injecting a first blood sample composed of blood taken from a patient (human or other mammal), and (b) a gas vent through which air displaced by injecting the first blood sample into the bay, can be expelled to accommodate the first blood sample. The gas vent comprises a porous plug through which air can be expelled upon injecting the first blood sample. Once air has been expelled through the porous plug, it generally seals against loss of the blood sample. The analyzer-unit is adaptable for testing a sample of blood from a patient to whom a drug is being administered, and therefore likely present in the patient's blood (e.g., an antiplatelet drug discussed, further, below). The analyzer-unit may be comprised of a plurality of bays, all in fluid communication with the same entry port for injecting a first blood sample composed of blood taken from a patient, and (b) a gas vent through which air displaced by injecting the first blood sample into the bay, can be expelled to accommodate the first blood sample. Alternatively, the analyzer-unit may be comprised of a plurality of bays, each bay being in fluid communication with a respective entry port and an associated gas vent through which air displaced by injecting a respective blood sample into the respective bay, can be expelled.

### BRIEF DESCRIPTION OF DRAWINGS & ATTACHMENT A

For purposes of illustrating the innovative nature plus the flexibility of design and versatility of the new system and associated technique set forth herein, the following background references and several figures are included. One can readily appreciate the advantages as well as novel features that distinguish the instant invention from conventional sensing systems and techniques. Where similar components are represented in different figures or views, for purposes of consistency, effort has been made to use similar reference numerals. The figures, as well as background technical materials, are included to communicate the features of applicants' innovative device and technique by way of example, only, and are in no way intended to limit the disclosure hereof. Any enclosure identified and labeled an ATTACHMENT, is hereby incorporated herein by reference for purposes of providing background technical information.

FIG. 1A is a depiction of the blood coagulation cascade: two pathways 10 result in the formation of fibrin (12), the building block of a hemostatic plug (i.e., clot). Further details about the blood coagulation cascade are shown in the diagram labeled ATTACHMENT B hereof, entitled The Coagulation Cascade, © 2003.

FIG. 1B depicts a conventional TEG plot 18 demonstrating a fibrinolysis stage. Parameters of interest include R the latency before clotting, K the clotting time, MA the maximum amplitude (clot strength), and α the rate of clot strengthening.

FIG. 2 shows representative TEG patterns 20 of blood corresponding to different clotting profiles, as labeled from top-to-bottom: Normal, Hypercoagulation, Platelet blocker, D.I.C. stage 1, D.I.C. stage 2, Fibrinolysis.

FIG. 3 is a high level schematic of a magnetoelastic sensor element 34 under-going interrogation through a magnetic field. The resonance spectrum 36 of the sensor is obtained by subtracting a background spectrum from the measured sensor response.

FIG. 4 is a graphical representation of the real and imaginary parts of the resonance spectrum (i.e., magnitude and phase as a function of frequency) obtained from a 12.5 mm×5 mm×28 μm magnetoelastic sensor element. The resonance frequency is defined as the frequency that corresponds to a max 40 of the real spectrum.

FIG. 5 is a high level schematic of a magnetoelastic sensor element (shown in cross-section fashion) oriented in a vertical position (left) and horizontal position (right).

FIG. 6 is a graphical representation of data collected with a magnetoelastic sensor element in both vertical and horizontal orientations (as depicted in FIG. 5): Sensor data taken while in a vertical position (upper graph 62) shows effectively no sign of a settling effect; whereas, the resonance amplitude of a horizontally oriented sensor element (lower graph 64) decreases exponentially, with time.

FIG. 7 is a high level flow diagram detailing core as well as additional steps of a technique 760 coined by applicants as a ‘frequency sweep’ (see, also, FIG. 16 of pending parent app. at 160) such as is performed by a computerized unit, e.g., that shown at 78 in FIG. 12 hereof.

FIG. 8 is a graphical representation of the reconstructed impedance spectrum of the sensor element after subtracting the coil impedance from total impedance of a coil combined with sensor element for the equivalent standard circuit model shown in FIG. 2 of pending parent app (see, also, FIG. 6 of pending parent app). This reconstruction is performed, for example, by employing the technique 930 represented by the flow diagram in FIG. 9, herein.

FIG. 9 depicts a method 930 of reconstructing the sensor impedance spectrum by subtracting the coil impedance from a total measured impedance of coil and sensor element (see, also, FIG. 3 of pending parent app at 30).

FIG. 10 is a high level block diagram depicting a system 100 of circuit elements (core as well as additional elements) for automatic implementation of the unique impedance analysis technique (see, also, FIG. 7 of pending parent app.) used by the invention in connection with the processing of emissions information obtained from sensing elements.

FIG. 11 is a high level block diagram depicting a system 70 of circuit elements (core as well as additional elements) for automatic implementation of the unique impedance analysis technique employed in connection with processing emissions information obtained from sensing elements. Please refer also to FIG. 10, hereof, at 100, and to FIG. 27 of pending parent app. at 200.

FIG. 12 is a high level schematic representing components of an embodiment of a magnetoelastic analyzer-unit 80 adapted for obtaining information from three different samples of blood 89a, b, c, each initially contained within a syringe/plunger-type mechanism, respectively at 86a, b, c; analyzer-unit 80 also includes a detection sub-unit 81 and a cartridge sub-unit 84 having three sensor elements 83a, b, c.

FIG. 13 is an isometric schematic representing components of an alternative magnetoelastic analyzer-unit 50 having elements 53a-d positioned within cartridge 54.

FIG. 14 is a high level schematic representing components of a cartridge unit 54 such as is shown in FIG. 13.

FIG. 15 is an isometric schematic (digital photo) representing components of the cartridge unit 54 represented in FIGS. 13 and 14.

FIGS. 16A-16B are isometric schematics (digital photos) representing components of an alternative cartridge structure 54′ similar to that shown in FIG. 15, but instead having a single bay/chamber 59a′.

FIGS. 17A-17B are high level schematics; FIG. 17A is a top plan view and FIG. 17B an end plan view representing components of an alternative cartridge unit 154, similar to that at 54 in FIG. 14, such as can be incorporated into analyzer-unit 50, FIG. 13.

FIG. 18 is a flow diagram detailing a method 140 for automatically determining a quantification for platelet contribution to clot formation in whole blood or platelet-rich plasma (PRP) using magnetoelastic sensor elements according to the invention.

FIG. 19 is a flow diagram detailing core as well as additional steps of a method 90 for automatically determining a quantification for platelet contribution to clot formation in whole blood or platelet-rich plasma (PRP) using magnetoelastic sensor elements.

FIG. 20 is a flow diagram detailing core as well as additional steps of a method 110 for automatically determining a quantification for platelet contribution to clot formation in whole blood or PRP using magnetoelastic sensor elements.

FIG. 21 graphically represents the normalized time dependent change in measured resonance amplitude of a magnetoelastic sensor element immersed in each of four blood sample mixtures.

FIG. 22 graphically represents ‘settling-compensated’ (i.e., the settling effect has been subtracted from data) normalized, time dependent change in measured resonance amplitude of magnetoelastic sensors immersed in the blood sample mixtures shown.

FIGS. 23-25: FIG. 23 is a clot profile of a bovine blood sample captured by a magnetoelastic sensor element. The clot profile is similar to the lower half of the TEG curve shown in FIG. 24. The curve in FIG. 23 is mirrored (by drawing another line with the same amplitude but opposite sign), and the resulting curve/shape, shown in FIG. 25 is analogous to FIG. 24.

FIGS. 26a,b show the TEG curves for three different blood concentrations (whole blood, 1:4 dilution and 1:8 dilution) measured by: FIG. 26a a Haemoscope TEG® analyzer, and FIG. 26b an analyzer-unit using magnetoelastic sensors.

ATTACHMENT A (8 pages) The Clotting Times, October 2004, incorporated by reference for the background technical discussion contained therein.

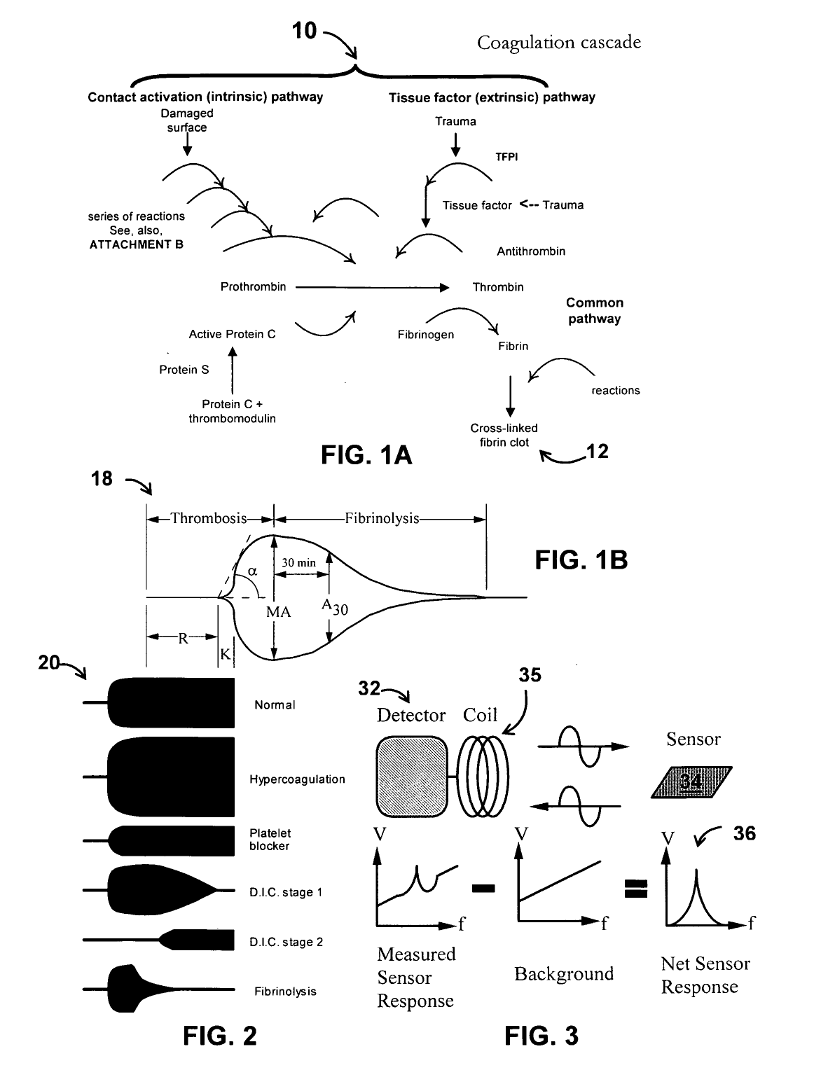

ATTACHMENT B (1 page) The Coagulation Cascade, © 2003 American Association for Clinical Chemistry, updated Feb. 19, 2004, incorporated by reference for further background technical information contained therein, see also FIG. 1A, hereof.

### DESCRIPTION DETAILING FEATURES OF THE INVENTION

By viewing the figures which depict representative structural embodiments, and associated process steps, one can further appreciate the unique nature of core as well as additional and alternative features of the new blood test system/unit, and associated technique/platform. Back-and-forth reference has been made to the various figures—schematics, graphical representations of functional relationships, and flow diagrams which, collectively, detail core as well as further-unique features—in order to associate respective features, for a better appreciation of the unique nature of the invention.

FIG. 1A is a depiction of the blood coagulation cascade: two pathways 10 result in the formation of fibrin (12), the building block of a hemostatic plug (i.e., clot); these pathways represent a series of chemical reactions as explained above. Further details about the blood coagulation cascade are shown in the diagram labeled ATTACHMENT B hereof, entitled The Coagulation Cascade, © 2003.

FIG. 1B is a familiar diagram; depicted is a conventional TEG plot/pattern 18 covering both the thrombosis and fibrinolysis stages. Introduced in 1981, the thromboelastograph (TEG) is a clinical test that provides one type of quantitative evaluation of the formation and strength of blood clots over time. It gives a high-level assessment of hemostatic function that helps in visualizing the whole coagulation process and its dynamics. The TEG test measures viscoelastic properties of blood undergoing a clotting process, revealing the time dependent kinetics of clot formation. The availability and relative proportion of various factors responsible for the clot formation can be evaluated by interpreting a resulting TEG pattern. The x-axis of a TEG plot/curve represents time and y-axis the clot strength. Generally, a TEG plot consists of two horizontal lines/curves, with the vertical separation distance therebetween representing blood clotting strength. When blood is liquid, the two lines join together. As the blood begins to clot, the lines split and gradually trace a ‘C’ shaped curve. In some cases, the clot breaks down after a period of time and the two lines rejoin. A TEG pattern/curve also reveals the strength and stability of the formed clot, thus provides some information about the ability of the clot to perform the work of hemostasis. Defects in the coagulation process or abnormalities in the platelet function are reflected in resulting TEG pattern which deviate from ‘normal’ or a standard, anticipated pattern shape (patterns at 20, FIG. 2).

Parameters of interest for TEG pattern 18 include: R the latency before clotting (the time to initial fibrin formation), K the clotting time, MA the maximum amplitude (clot strength), and α the rate of clot strengthening. To health care providers and testing laboratories that regularly test patient blood, TEG plots (as labeled with variables R, K, MA, α, and so on) are familiar, as are the shapes in FIG. 2, which shows representative TEG patterns 20 of blood corresponding to different clotting profiles, as labeled from top-to-bottom: Normal, Hypercoagulation, Platelet blocker, D.I.C. stage 1, D.I.C. stage 2, Fibrinolysis. In comparison with the TEG test, the erythrocyte sedimentation rate (ESR) is a non-specific screening test that measures the settling rate of red blood cells. Since many diseases such as hemophilia, von Willebrand disease, polymyalgia rheumatica, temporal arteritis, some types of cancer, and anemia directly affect the clotting process and blood cell counts, TEG and ESR analyses can provide valuable information to a health care provider.

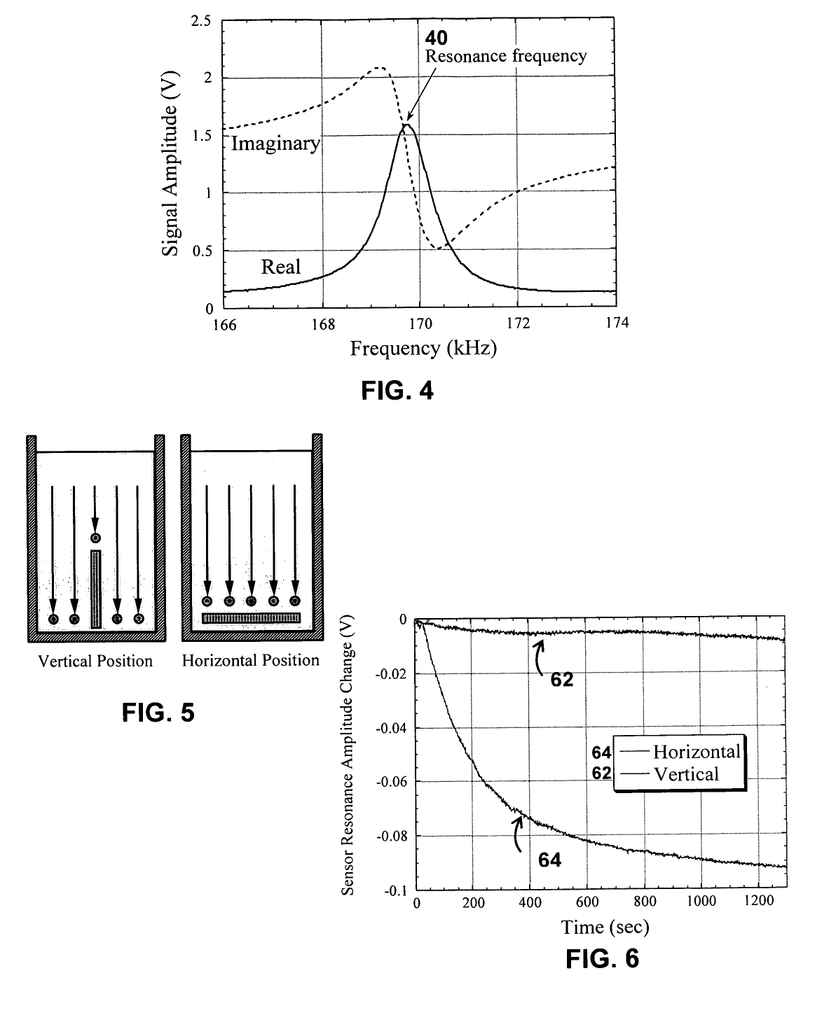

FIG. 3 is a high level schematic representing a magnetoelastic sensor element 34 under-going interrogation through exposure of a magnetic field. A detector 32 and interrogation coil 35 interoperate, as explained in applicants' prior work, to produce emissions that are measured. The resonance spectrum 36 of the sensor is obtained by subtracting a background spectrum from the measured sensor response. While a sensor element 34 may be monitored through a transient frequency-counting process or via fast Fourier transformation operation, another way to measure the sensor response is by capturing the frequency-domain resonance spectrum. To capture the resonance spectrum of the sensor, the detector first sends a frequency varying, constant amplitude current to a magnetic coil to generate a magnetic AC field. When the sensor resonates, it generates a magnetic flux that induces a voltage on the same coil. As a result, the resonance spectrum of the sensor is also embedded in the voltage across the magnetic coil. To obtain the sensor resonance spectrum as shown in FIG. 4, the background voltage across the coil is first measured in the absence of the sensor, and the measured voltage (with the sensor) is subtracted from the background measurement (see FIG. 3). For ease of use, once the background spectrum has been determined, it is preferably stored in the device memory for future use.

FIG. 4 is a graphical representation of the real and imaginary parts of the resonance spectrum (i.e., magnitude and phase as a function of frequency) obtained from a 12.5 mm×5 mm×28 μm magnetoelastic sensor element. The resonance frequency is defined as the frequency that corresponds to the maximum point 40 of the real spectrum. The resonance amplitude of magnetoelastic element emissions is dependent on the mass loading and elasticity of a coating placed atop the element, just as emissions resonance frequency is (as reported by applicants in their earlier work). Since mass loading dampens the amplitude of vibration, it decreases the measured voltage amplitude of the sensor. Similarly, the elasticity of a coating atop a sensor element is proportional to the resonance amplitude.

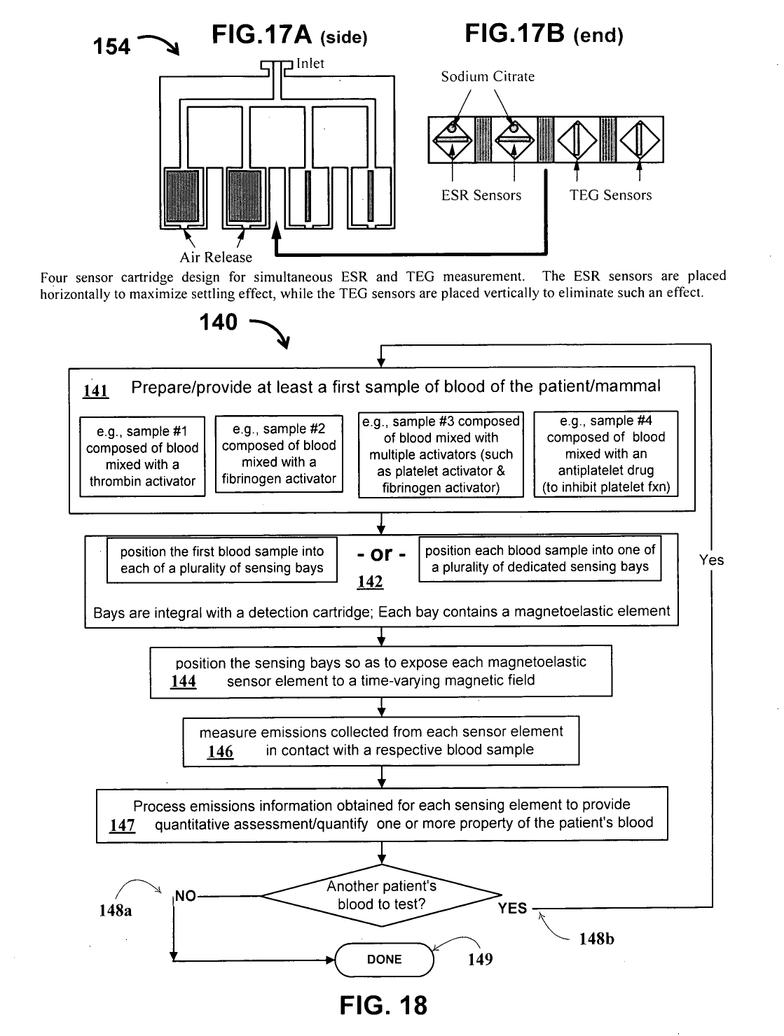

FIG. 5 is a high level schematic of a magnetoelastic sensor element (shown in cross-section fashion) oriented in a vertical position (left) and horizontal position (right). According to the invention, to obtain information about a blood sample from the emissions measured from a respective sensor element in contact therewith, the sensor element is preferably oriented in a horizontal fashion (right-hand graphic) where a maximized response to sedimentation within the blood sample is sought (for example, where the element is targeted for taking a measurement of Erythrocyte Sedimentation Rate, ESR). Orienting the sensor element vertically (left-hand side graphic) allows for TEG analysis, without seeing effects of sedimentation as preferred in that case. See, also, the discussion in connection with FIGS. 17A and 17B showing a cartridge with an inlet to receive a blood sample: The ESR sensor elements are oriented horizontally (to maximize the settling effect); and the TEG sensor elements are oriented vertically (to minimize such an effect).

FIG. 6 is a graphical representation of the change in resonance amplitude over time of emissions collected with a magnetoelastic sensor element in two different orientations, vertical and horizontal (as depicted in FIG. 5), when immersed in citrated bovine blood, by way of example. Sensor data taken while in a vertical position (upper graph 62) shows effectively no sign of a settling effect: The deviation—only a slight decline of change in amplitude over time—observed when the element is vertically oriented is due to lack of temperature compensation during test. Whereas, one can appreciate that the resonance amplitude of a horizontally oriented sensor element (lower graph 64) decreases exponentially, with time. Thus, the settling effect can be minimized or maximized by changing sensor orientation within a cartridge sensing bay/chamber.

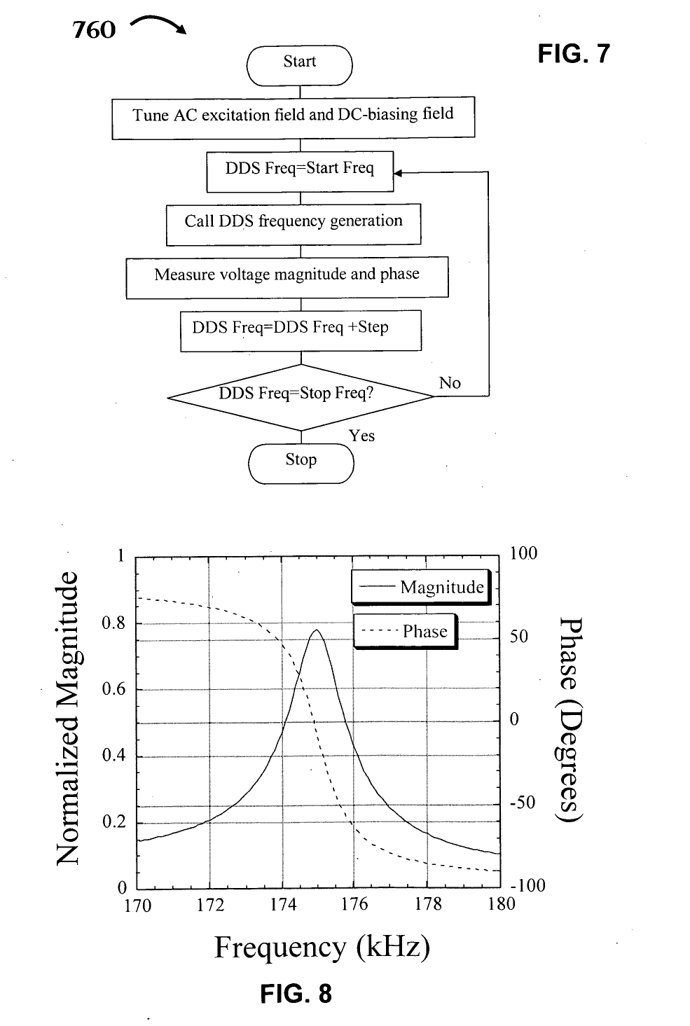

FIG. 7 is a high level flow diagram detailing core as well as additional steps of a technique 760 coined by applicants as a ‘frequency sweep’ (see, also, FIG. 16 of pending parent app. at 160) such as is performed by a computerized unit, e.g., microcontroller/microprocessor unit 78, FIG. 12 hereof, for obtaining measurements from a coil unit such as that represented by the block labeled 35, 34 in system circuit diagram 100 of FIG. 10. Correspondingly numbered are: coil 35 in proximity to sensor element 34 of FIG. 3. See, also, the feature labeled 15/10 in FIG. 1 of pending parent app. depicting the excitation of the coil unit 15/10 so as to collect measurements for reconstructing an impedance spectrum of one or more sensor element(s).

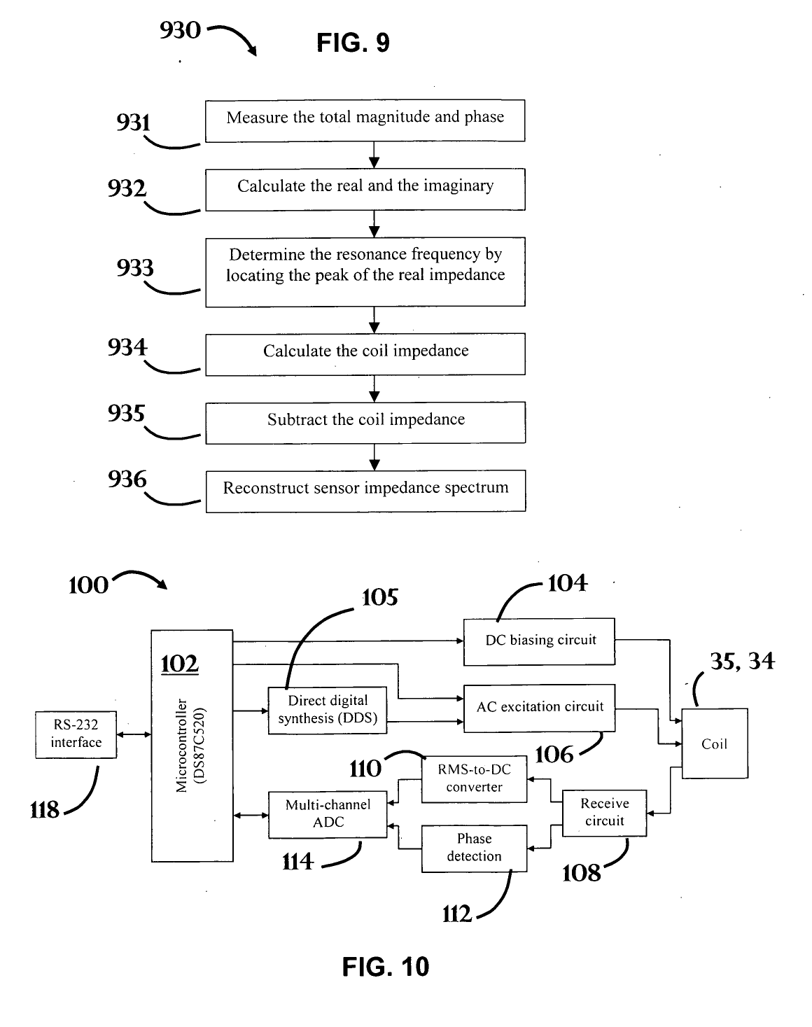

FIG. 8 (see, also, FIG. 6 of pending parent app.) is a graphical representation of a reconstructed impedance spectrum of the sensor element after subtracting the coil impedance from total impedance of a coil combined with sensor element for the equivalent standard circuit model shown in FIG. 2 of pending parent app. This reconstruction is performed, for example, by employing the technique 930 represented by the flow diagram in FIG. 9, herein.

FIG. 9 depicts a method 930 of reconstructing the sensor impedance spectrum by subtracting the coil impedance from a total measured impedance of coil and sensor element (see, also, FIG. 3 of pending parent app at 30). As stated above, the graphical representation in FIG. 8 is of a reconstructed impedance spectrum of the sensor element after subtracting the coil impedance from total impedance of a coil combined with sensor element for the equivalent standard circuit model shown in FIG. 2 of pending parent app.

FIG. 10 is a high level block diagram depicting a system 100 of circuit elements (core as well as additional elements) for automatic implementation of the unique impedance analysis technique (see, also, FIG. 7 of pending parent app.) used by the invention in connection with the processing of emissions information obtained from sensing elements.

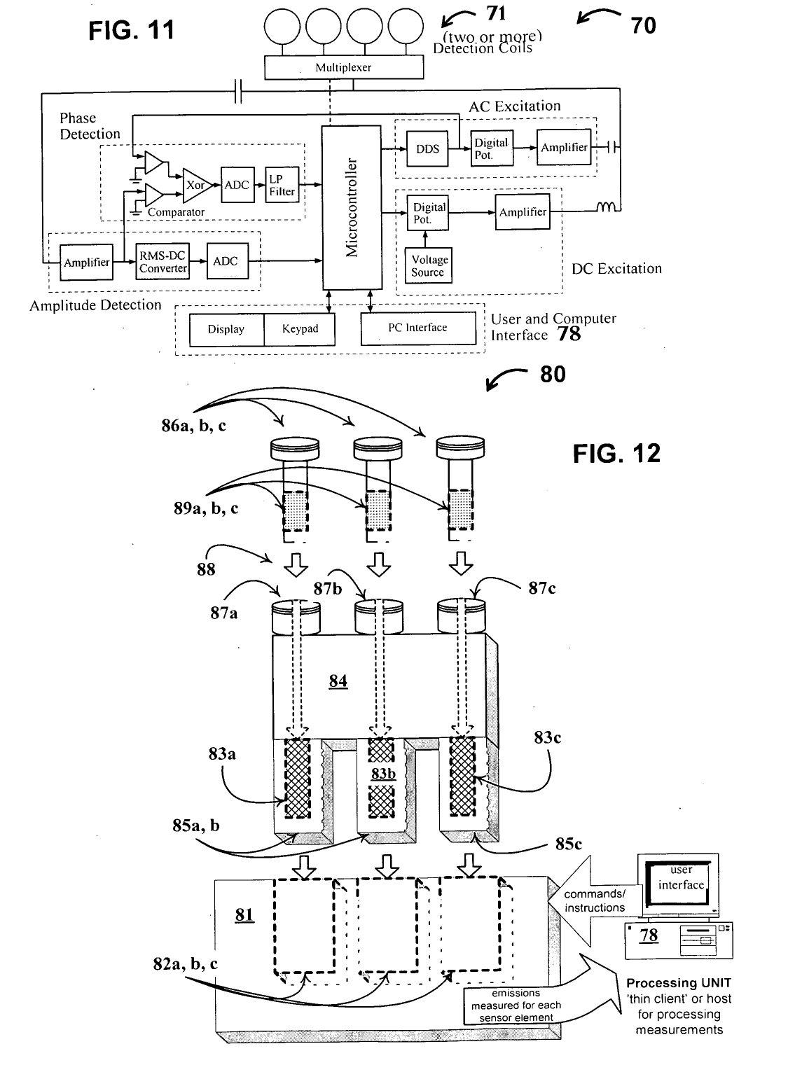

FIG. 11 is a high level block diagram depicting a system 70 of circuit elements (core as well as additional elements) for automatic implementation of the unique impedance analysis technique employed in connection with processing emissions information obtained from sensing elements. Please refer also to FIG. 10, hereof, depicting system 100, and to FIG. 27 of pending parent app., system 200. In FIG. 11, system 70 includes sensing analyzer-unit circuitry having six main functional components (identified in-phantom): microcontroller, amplitude detection, phase detection, DC excitation, AC excitation, and user and computer interface. A multiplexer is preferably used to connect the circuit to one of the plurality of detection coils 71. The multi-sensor unit 70 shown by way of example, here, features detection coils 71 (four are shown) for simultaneous monitoring of the responses of a respective number—two, three, four, and so on—sensor elements.

As shown in FIG. 11, the multi-sensor unit circuitry includes a Multiplexer implemented to connect the circuit to one of the four detection coils during the measurement of emissions from the sensor array. As labeled, a Microcontroller oversees operations of the system. It instructs the AC and DC excitation circuits to generate the excitation fields, as well as processes the captured sensor response. It controls the user interface and communicates with the PC, and also the multiplexer. The AC Excitation circuit consists of a direct digital synthesis (DDS) chip for generating a precise AC signal, which is sent to an amplifier and then the excitation coil. A controllable digital potentiometer is shown, here, and operates to tune the AC excitation voltage, thus changing the excitation field strength. A capacitor is shown, here, to isolate the AC excitation circuit from the DC current generated by the DC excitation circuit. The DC Excitation circuit uses a voltage source to generate the DC current. A potentiometer is shown, here, to control the DC current magnitude and hence the biasing field. An inductor is shown, here, to isolate the DC excitation circuit from the AC current.

FIG. 12 is a high level schematic representing components of an embodiment of a magnetoelastic analyzer-unit 80 adapted for obtaining information from—for example as shown in this embodiment—three different samples of blood identified as 89a, b, c; each sample is initially hermetically contained within syringe/plunger-type mechanism, respectively, 86a, b, c, to protect it from outside contamination. Analyzer-unit 80 also includes a detection sub-unit (labeled 81) and a cartridge sub-unit (cartridge assembly at 84) having three bays/chambers 85a, b, c, each comprising a respective sensor element 83a, b, c. Each bay 85a, b, c is shaped and sized to fit into a respective cavity area 82a, b, c within the interior spacing of a respective coil (coils not shown, for simplicity) of a housing for the detection sub-unit 81.

Each blood sample 89a, b, c composed of blood taken from a patient (any mammal, including humans and non-human animals) is inserted (along arrow 88) into a respective receiving port 87a, b, c of cartridge assembly 84 which is in communication with a respective bay 85a, b, c within which a sensor element 83a, b, is located. As depicted here, each syringe 86a, b, c is initially ‘loaded’ with a particular blood sample 89a, b, c. As explained more-fully elsewhere herein, each blood sample 89a, b, c is composed of blood from a patient mixed with one or more additive, such as a thrombin activator, a fibrinogen activator, platelet activator, an antiplatelet drug (which might already have been administered to the patient before drawing the blood therefrom). While three bays are depicted in FIG. 12 by way of example, if more bays are fabricated integral (e.g., molded) with cartridge 84, additional blood samples composed of the patient's blood mixed with a different activator/agent, can be analyzed. As explained in applicants' earlier work—and further below—energy emitted from a magnetoelastic element exposed to a time-varying field is related to the size of the element and the analyte undergoing analysis (in this case, the blood sample).

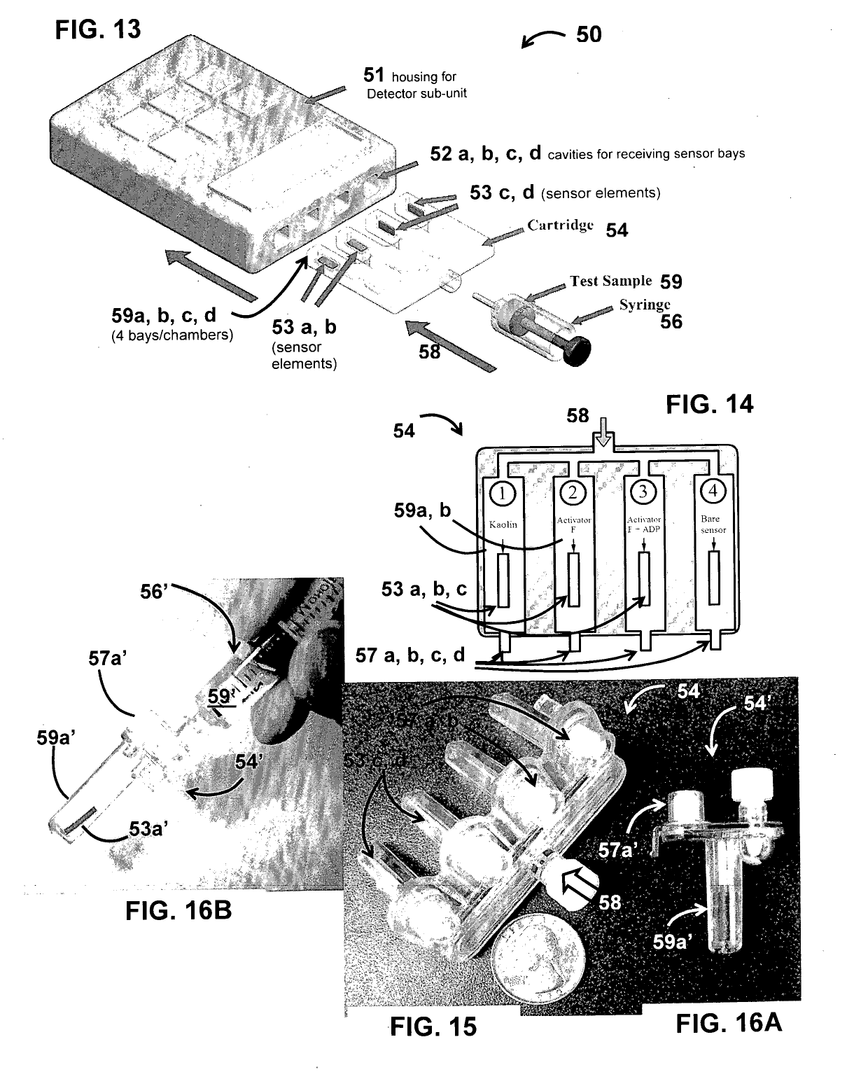

Once each bay 85a, b, c is positioned into a cavity area 82a, b, c within a respective coil (not shown for simplicity) undergoing excitation so as to create a time-varying magnetic field, emissions are measured from each magnetoelastic sensor element 83a, b, c in contact with a respective blood sample. In operation, emissions are measured from the first magnetoelastic sensor element 83a to provide first information relating to a property of the blood in sample 89a; emissions are also measured from the second sensor element 83b to provide second information relating to a property of the blood in second sample 89b, as are emissions measured from the third sensor element 83c to provide third information relating to a property of the blood in third sample 89c. Jumping to alternative embodiment shown in FIGS. 13-16: emissions are likewise measured that emanate from the sensor elements 53a, b, c, d as well as that labeled 53a′ (FIG. 16B). The information obtained from emissions respectively measured from each sensor element is uniquely processed to provide at least one quantitative assessment is made of the blood, as further explained herethroughout.

The measuring of emissions to obtain information about the blood in a respective sample, is preferably accomplished by employing one or more of the techniques co-developed by applicants hereof, such as any suitable technique described and referenced in applicants' co-pending parent application Ser. No. 11/710,294. While co-pending application Ser. No. 11/710,294 is directed to an impedance analysis technique applied to measure steady-state vibration of a magnetoelastic sensor element forced by a constant sine wave excitation, the co-pending parent application Ser. No. 11/710,294 also references an earlier technique, namely, the threshold-crossing counting technique invented by three co-applicants hereof (Drs. K. Zeng, K. G. Ong, and C. A. Grimes) and detailed in U.S. Pat. No. 7,113,876 for “Technique and Electronic Circuitry for Quantifying a Transient Signal using Threshold-crossing Counting to Track Signal Amplitude.” As further detailed in applicants' co-pending parent application Ser. No. 11/710,294 and the earlier-filed (now granted) U.S. Pat. No. 7,113,876 directed to threshold-crossing counting technique, one can measure resonance frequency of sensor element emissions, Q of the resonance, or amplitude of the resonance. Alternatively, as explained by applicants earlier, one can set and select an initial (‘listening’) frequency and measure the amplitude at this initial, listening frequency. Listening frequency, in this case, is not synonymous with sensor element resonance frequency, as resonance shifts with whatever is happening within blood sample, e.g., clotting, to change its viscosity over time.

As explained in parent application Ser. No. 11/710,294: An electronic implementation of the impedance analysis technique can, for example, include a single circuit board that, when interfaced with a processor unit (e.g., within a palmtop, laptop, handheld, remote hard-wired, remote wireless, and so on), uses a solenoid coil unit to characterize sensor resonance behavior in the frequency domain, after having obtained the complex (magnitude, phase) impedance spectrum of the sensor element from a measured impedance (a ‘combined’ impedance for the system of sensor element plus coil); see, also, FIG. 10 at 100 and FIG. 11 at 70, hereof, as well as associated FIGS. 3, 4, 7, 8, 9 depicting steps and graphical representation(s) related to measuring emissions from a sensor element to provide information about the analyte (sample) being analyzed. As explained in Zeng et al., U.S. Pat. No. 7,113,876—incorporated herein by reference for its technical background—the threshold-crossing counting technique measures free vibration of a sensor element once excitation of the element has stopped. The Zeng et al. U.S. Pat. No. 7,113,876 technique includes a threshold comparison feature employing the transient signal received (which had been emitted as a result of the sensor element vibrations), coined ‘threshold-crossing counting. While applicants’ threshold-crossing counting technique is useful in a wide range of environments, the newer impedance analysis technique can provide superior results, especially in viscous environments where the medium through which sensor emissions must ‘ring’ in order to provide sensor information, is viscous.

The magnetoelastic sensors are preferably made from elongated magnetostrictive ferromagnetic amorphous alloys (see for example, Vacuumschemaltze Corporation, distributor of a suitable sensor material) that generate both longitudinal elastic waves and magnetic flux when exposed to a time varying magnetic field. The elastic waves can be detected by a microphone (audio sensor pick-up device) while the magnetic flux can be sensed by a remotely placed inductive pick-up coil. The resonance frequency of the magnetoelastic wave depends on the Young's modulus of elasticity of the sensor (E), density (ρₛ), the Poisson ratio (σ), and length (L) of the sensing element. Mathematically, the fundamental resonance frequency f₀ of the elastic vibrations is expressed as:

$${f}_{0} = \frac{π}{L} \sqrt{\frac{E}{{ρ}_{s} ( 1 - {σ}^{2} )}} \qquad ( 1 )$$

For a specific magnetoelastic material, E, ρₛ, and σ remain constant, hence the resonance frequency can be varied by changing the length of the sensor element. For the Vacuumschemaltze material the resonance frequencies of illustrative 6 mm wide 28 μm thick sensors in air, 12 mm and 15 mm length, are approximately 180 kHz and 145 kHz respectively.

When an elongated magnetoelastic sensor element is immersed in a liquid the viscosity of the surrounding medium acts as a damping force to the sensor oscillations that result in a downward shift of the resonance frequency, which is expressed as:

$$Δ f = - \frac{\sqrt{π {f}_{0}}}{2 π {ρ}_{s} d} {( η {ρ}_{l} )}^{1 / 2} \qquad ( 2 )$$

Where f₀ is the resonance frequency of the sensor in air, ρₛ and d the density and thickness of the sensor, and ρₗ and η the density and viscosity of the liquid, respectively. This implies a change in liquid density and/or viscosity results in a corresponding shift in the resonance characteristics of a liquid immersed magnetoelastic sensor. The (ηρᵢ)^{1/2} term arises from the wave equation describing the propagation of shear waves in a liquid. The effect of liquid density ρₗ arises from the force=mass×acceleration term, while liquid viscosity η appears as a drag term. The shift in resonant frequency is proportional to the square-root of ηρₗ as the wave equation contains the square of the wave velocity.

Although Eqn. (2) explains the behavior of a magnetoelastic sensor in a liquid of changing viscosity, such as a blood sample undergoing a clotting cascade, it does not fully explain the change in sensor characteristics when the sensor is mass-loaded. It has been shown that when a small mass Δm is loaded on the surface of a magnetoelastic sensor of mass m₀, the shift in resonance frequency Δf is given by:

$$Δ f = - {f}_{0} \frac{Δ m}{2 {m}_{0}} \qquad ( 3 )$$

where f₀ is the resonance frequency without mass any mass loading. Eqn. (3) quantifies the change in resonance frequency due to mass loading and is particularly useful is describing the sensor behavior in blood samples due to settling of red blood cells or aggregated platelets.

However, Eqn. (3) does not take into account the elastic stress in the applied mass load. Considering a uniform mass adhered to the sensor surface the rate and sign of the frequency change due to the mass coating depends on the elasticity and density of the coating in comparison with that of the sensor. If m₀ and mₜ are the mass of the sensor and the total mass after coating, the ratio of the measured frequencies before (fₒ) and after (f) applying a coating is:

$$\frac{f}{{f}_{0}} = \sqrt{\{ \frac{{m}_{0}}{{m}_{t}} + \frac{{E}_{c} / {ρ}_{c}}{{E}_{s} / {ρ}_{s}} ( 1 - \frac{{m}_{0}}{{m}_{t}} ) \}} \qquad ( 4 )$$

E_{c} and Eₛ are the modulus of elasticity of the coating and the sensor, respectively, and ρ_{c} and ρₛ are the density of the coating and the sensor, respectively. Eqns. (3) and (4) describe the overall behavior of a magnetoelastic sensor immersed in a complex liquid, blood (considered an ‘infectious material’ and on occasion referred to as a non-Newtonian liquid), taking into account effects settling, e.g. blood cells and platelets falling onto the sensor surface, and clot formation where the sensor is encased in a solid-like substance.

Similar to the resonance frequency, the resonance amplitude of a magnetoelastic sensor is also dependent on the mass loading and elasticity of the coatings. Since mass loading dampens the amplitude of vibration, it decreases the measured voltage amplitude of the sensor. In most cases the percentage change in voltage amplitude is an order of magnitude greater than the corresponding frequency shift; thus for applications such as measuring blood clotting characteristics—as uniquely done here—the resonance amplitude, instead of resonance frequency, can be measured as a function of time.

Magnetoelastic sensors have been employed by applicants in earlier work in a number of sensing applications through the tracking of the systematic variation of the resonance frequency and resonance amplitude of the sensor. As mentioned, various physical parameters such as temperature, pressure, liquid density and viscosity, fluid flow velocity, and thin film elastic modulus have been quantified using magnetoelastic sensors. In combination with analyte-responsive coatings, magnetoelastic sensors have been used as chemical sensors for pH and glucose, as gas sensors. As mentioned by applicants in their co-pending parent app., magnetoelastic bio-sensors have been used for the quantification of E.-Coli 0157:H7 bacteria, Staphyloccocal enterotoxin B, avidin, trypsin, and ricin.

According to one aspect of the present invention, the extent of clot formation in whole blood due to thrombin and fibrin generation and platelet aggregation has been measured by tracking the time dependent change in the sensor vibration amplitude under respective clotting conditions. Although Bachas, et al. (2004) mentioned use of magnetoelastic sensors to monitor blood clot formation, it was through subsequent work by applicants, mentioned elsewhere herein, whereby a compact microprocessor based magnetoelastic sensor system was produced based on a time domain analysis technique. The microprocessor based electronics enable characterization of sensor resonance characteristics in ≈10 ms, with a measurement resolution of a few Hz. The instant sensing platform is useful for measuring activated clotting time (ACT), as well as determination of Erythrocyte Sedimentation Rate (ESR), and Thromboelastograph (TEG) analyses of whole blood.

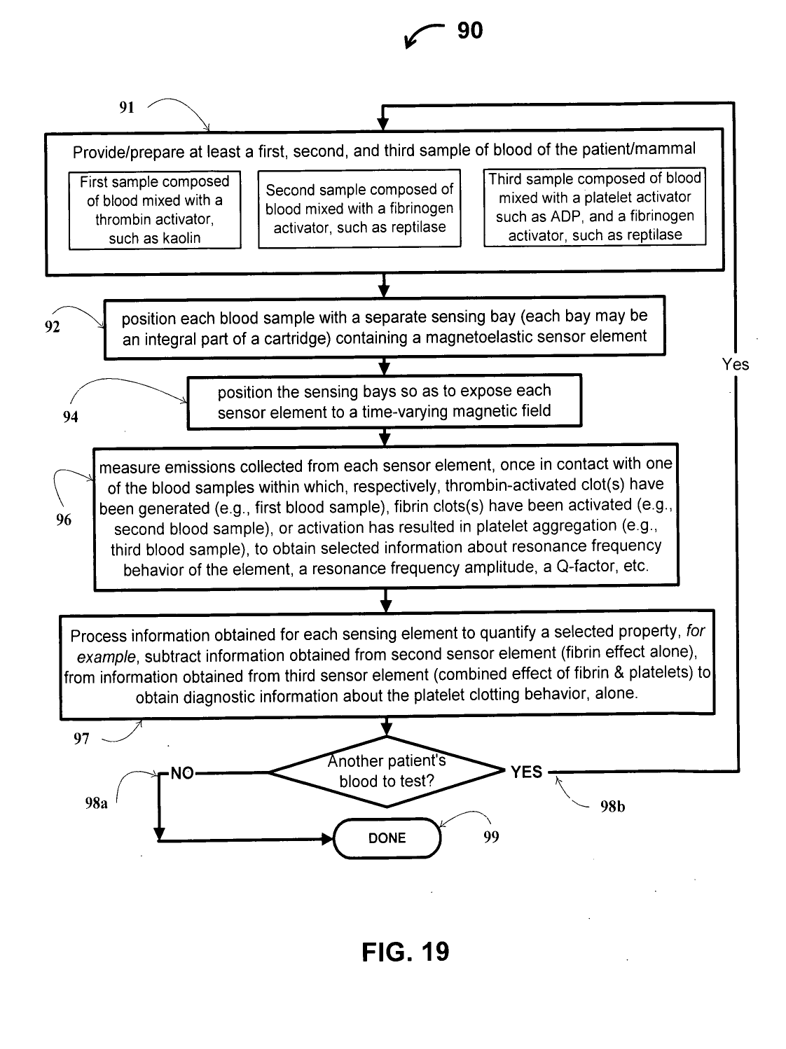

For this aspect of the invention—see, for example, FIG. 19 at 91—a first blood sample can be composed of a mixture of a selected amount of the mammal's blood to which kaolin has been added. Kaolin is an agent known to activate a ‘full’ clotting cascade—one that is initiated by thrombin generation—which is generally distributed in the form of a powered clay. The full clotting cascade can also be activated by mixing the blood with diatonaceous earth, one such mixture additive is distributed under the brand name CELITE™. A second blood sample can be composed of the mammal's blood to which reptilase has been added. Reptilase, an enzyme found in snake venom, functions to activate the fibrinogen to fibrin conversion (an agent in the formation of fibrin networks within a blood clot) distributed under brand names such as Batroxobin™ (from Pentapharm) and Activator F™. A third blood sample is composed of a selected amount of the mammal's blood to which reptilase and adenosine di-phosphate, ADP, have been added. ADP is a known platelet activator in the formation of blood clots. A clot formed by a fibrinogen activator such as reptilase along with a platelet activator such as ADP is generally considered mechanically ‘weaker’ than a clot developed using a thrombin activator such as kaolin.

As identified herein, in order to distinguish the contributions of thrombin and of fibrin in the clotting cascade of hemostasis, isolation and quantification of platelet activity is necessary. With thrombin mediated clotting, which resembles a normal clotting cascade (see, for example, FIG. 1A), clotting is initiated by treating blood with kaolin resulting in maximum hemostatic activity, with contributions from both the fibrin networks as well as platelet activation, and subsequent formation of a robust clot. Characterizing the clotting due only to the fibrin networks helps to isolate and quantify the platelet activity. To generate a clot based only on fibrin mediated clotting a heparinized sample of whole blood is treated with Batroxobin™, a proteolytic enzyme from Bothrops atrox venom. In contrast to thrombin, which releases the fibrinopeptides A and B from fibrinogen, Batroxobin™ specifically splits off fibrinopeptide A and does not affect other hemostasis proteins and platelets.

Turning to FIG. 13, this isometric schematic represents components of an alternative embodiment of a magnetoelastic analyzer-unit 50. Sensor elements 53a, b, c, d are shown positioned within cartridge 54 (may be made of Plexiglas® or other suitable moldable, bio-compatible material, such as MABS, Methyl-methacrylate Acrylonitrile Butadiene Styrene plastic material). In this embodiment, a single blood sample 59 (may be composed of blood taken from a patient to whom a particular drug is being directly administered, or not, and further mixed with a reagent/activator as explained elsewhere) is injected (generally in the direction labeled for reference at 58) into cartridge 54 using a syringe/plunger-type mechanism 56. The bays/chambers 59a, b, c, d of cartridge 54 are oriented and inserted (likewise in a general direction 58) into respective slots/cavities 52a, b, c, d, which each represent the spacing within adjacently located pickup coils (not shown in FIG. 13 for simplicity, and are oriented in side-by-side fashion with coil axes in parallel) located within a housing 51 of a detector sub-unit. One coil may be used to detect sensor emissions from each element 53a, b, c, d or separate coil windings (in electrical communication) are used, the axis of each to coincide with that of a respective cavity 52a, b, c, d within unit 51 (see also, FIG. 12, at 82a, b, c within unit 81).

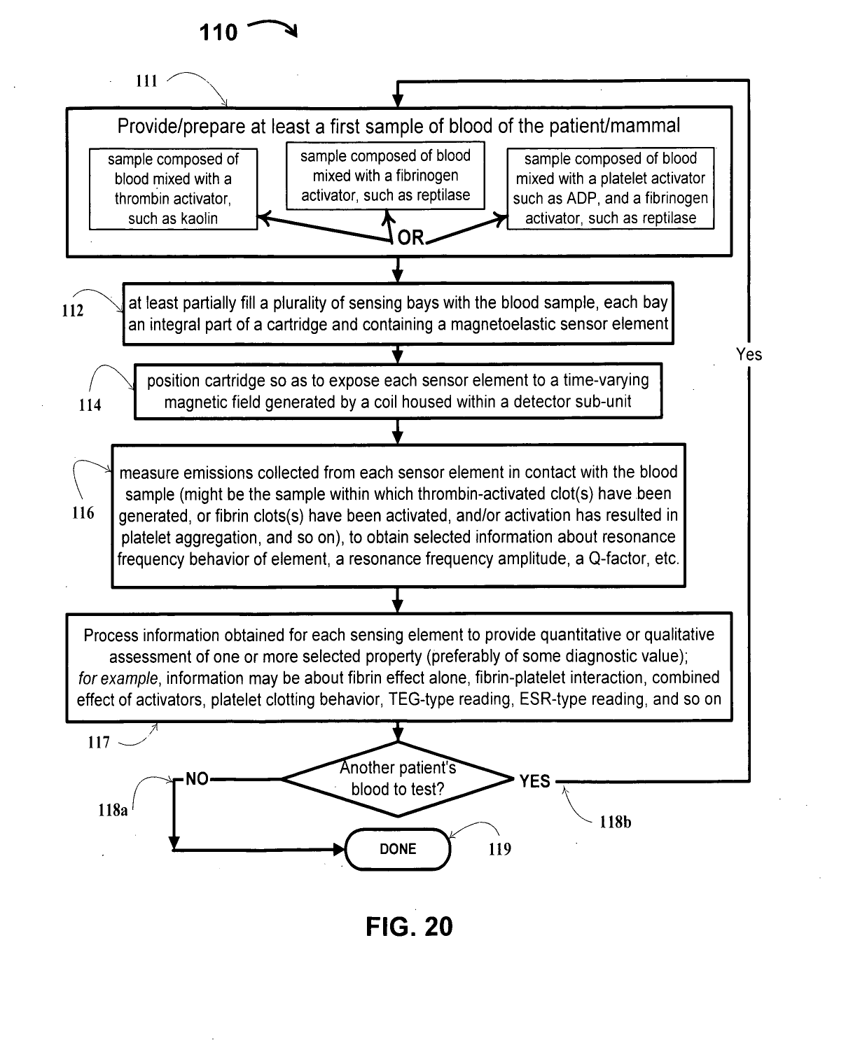

Blood sample analysis is carried out according to the unique technique set forth diagrammatically in more-detail in FIGS. 18 and 20. Analyzer-unit 50 of FIG. 13—by way of example only—is shown having four sensor elements 53a, b, c, d inter-connected by way of a fluid channel (adapted to accept the liquid blood so as not to come in contact with outside contaminants) to test a sample of blood 59. FIG. 14 is a high level schematic representing components of cartridge unit 54 for analyzer-unit 50 of FIG. 13. A blood sample entering at 58 flows into each of the bays 59a, b, etc., for contact with a respective sensor element 53a, b, c, for analysis thereof. In an alternative embodiment, prior to entering a respective bay 53a, b, c the sample liquid may be closed-off from the entry port 58 at locations \textcircled{1}, \textcircled{2}, \textcircled{3}, \textcircled{4}, and infused with one or more additive/activator, identified left-to-right by way of example in FIG. 14 as Koalin, Activator F, Activator F+ ADP, Bare sensor (no additive). Whether bays are infused with an activator at locations \textcircled{1}, \textcircled{2}, \textcircled{3}, \textcircled{4}, or flow into each bay 59a, b, etc., remains unrestricted so that effectively the same sample-mixture comes in contact with the various sensor elements 53a, b, c: respective exit ports are shown and labeled for each bay, as 57a, b, c, d through which air or other gas, displaced by injecting the liquid sample into the bay, is expelled (‘forced out’) to accommodate space for the liquid within the bay.

By way of example, a cartridge device built according to that depicted in FIG. 14 has an inlet 58 into which a blood/PRP sample is injected: One sample containing kaolin to generate a thrombin activated clot is injected into one of the bays; a second sample containing Activator-F (reptilase, an enzyme found in snake venom, such as that sold under the brand name Batroxobin™) to generate a fibrin clot is injected into a second of the bays, a third sample containing Activator-F (also, Act-F) and ADP to generate a fibrin+platelet aggregation plot is injected into a third bay, and the fourth bay/chamber can be reserved for a sensor element to collect data so that information collected by the other sensor elements can be adjusted/calibrated for settling, as needed.

A gas vent device, examples of which are shown in greater detail in FIG. 15, is positioned at each exit port 57a, b, c, d (see, also, FIGS. 16A and 16B at 57a′). The gas vent preferably comprises a porous plug through which air can be expelled when the liquid sample is injected into the bay. Once the displaced air has been expelled through the porous plug, preferably it seals against loss of the liquid blood sample (preferably designed as a gas permeable, yet liquid impermeable, membrane). To gain an appreciation of relative size of cartridge 54, by way of example only, a U.S. coin (25 cent-quarter) is shown in FIG. 15 next to cartridge 54.

The alternative cartridge structure 54′ shown in FIGS. 16A-16B is similar to that shown in FIG. 15, however, cartridge 54′ has a single integral bay/chamber 59a′ in which a sensor element 53a′ has been placed for analysis of a sample. A syringe 56′ containing a test blood sample 59′ (or other bio-analyte of interest, say, another body fluid) is used to inject the sample into the bay/chamber 59a′ vented by device 57a′ (gas permeable to allow air to escape, while holding back the test sample liquid). One can appreciate that cartridge structures 54, 54′, 154, may be made to be disposable.

FIGS. 17A-17B are high level schematics; FIG. 17A is a top plan view and FIG. 17B an end plan view representing components of an alternative cartridge unit 154, similar to that at 54 in FIG. 14, such as can be incorporated into analyzer-unit 50, FIG. 13. As shown, the analyzer-unit accommodates multiple sensor elements—two, three, four, and so on—which can be operating simultaneously, with the time-dependent frequency and amplitude responses of these sensors recorded to derive the TEG and ESR profiles. Orientation of the sensor elements shown in FIGS. 17A-17B are further explained in connection with FIG. 5, a high level schematic of sensor elements shown in cross-section fashion oriented in a vertical position (left) and horizontal position (right). As explained above, FIG. 6 graphically illustrates data collected with a magnetoelastic sensor element in both vertical and horizontal orientations: Sensor data taken while in a vertical position (62) shows effectively no sign of a settling effect; whereas, the resonance amplitude of a horizontally oriented sensor element (64) decreases exponentially, with time. The four-sensor configuration represented can accommodate simultaneous ESR and TEG measurements. The ESR sensor elements are preferably oriented horizontally (left-hand side of FIG. 17B) to capitalize on the settling effect, while the TEG sensor elements (right-hand side of FIG. 17B) are preferably oriented vertically—or 90-degrees (i.e., orthogonally) from orientation of the ESR elements—to minimize (or even eliminate) the settling effect. The blood samples used in connection with the ESR-dedicated sensor elements, may also be activated with an anti-coagulant, sodium citrate, to prevent blood from clotting during the determination of ESR.

The cartridge can be fabricated to accommodate one, two, three, four, five, six, seven, and so on, sensor elements—whether each element is sized and calibrated to collect information about one or more sample of patient's blood—according to the following structural embodiments, among others:
(a) several different sample-mixtures comprising the blood (with or without mixing-in one of a wide variety of additives/activators) such as is detailed in FIG. 12 at 89a, b, c, (where each syringe 86a, b, c is initially ‘loaded’ with a blood sample 89a, b, c) and is suggested schematically in FIG. 14 (where additives/activators are injected at a location \textcircled{1}, \textcircled{2}, \textcircled{3}, \textcircled{4}, respectively, of bays 59a, b, etc., after a sample of blood has been injected 58 into the cartridge 54);
(b) one sample-mixture 59 comprising blood (with or without mixing-in one or more of a wide variety of additives/activators) such as is detailed in FIG. 13, where each sensor element 53a, b, c, d can be dedicated (sized and calibrated) to test and provide information concerning a parameter/property of the blood;
(c) pairs of ‘redundant’ of measurements are made using one sample-mixture of blood 59, such as where two elements, say, 53a and 53b are dedicated (oriented, sized, and calibrated) to test a similar parameter/property (e.g., TEG profile concerning clot strength), and two other elements, say, 53c and 53d are dedicated (sized and calibrated) to test another parameter (e.g., make ESR readings)—as suggested schematically at 154 in FIGS. 17A-17B. Note that, where a cartridge (such as is shown at 54, 154) has the capability to make redundant measurements of a blood sample (e.g., pairs, triplets, and so on), an average reading/output is displayed for each desired parameter reading; furthermore, in this embodiment, redundant readings that clearly fall outside of an expected or anticipated threshold range of values difference can be discarded (false reading). In the event both the information obtained from measuring emissions from one sensor element (e.g., a TEG sensor) and that obtained from measuring emissions from a second TEG sensor element, fall outside an anticipated threshold range, the analyzer-unit is preferably programmed to not process a TEG measurement, but rather, communicate that an error in reading, etc., has occurred.

For case (c) contemplated above, steps may include: measuring first emissions collected from sensor element 53a/53a′ while in contact with a sample of blood; measuring second emissions collected from another of the sensor elements while in contact with the sample of blood, both sensor elements having been calibrated (sized and shaped) to provide a first type of information (for example, as suggested in FIGS. 17A, 17B, a TEG plot/pattern); measuring third emissions collected from another of the sensor elements in contact with a sample of the blood, and measuring forth emissions collected from yet another of the sensor elements, the third and fourth sensor element calibrated to provide a second type of information (for example, as suggested in FIGS. 17A, 17B, redundant ESR assessments). The information obtained from measuring the first emissions can be compared with that obtained from measuring the second emissions to process a first quantification for the blood. Any information/values that fall outside of an anticipated threshold value for the first type of information, are preferably disregarded and not used when determining the first quantification (e.g., TEG plot). Likewise, information obtained from measuring the third emissions can be compared with that obtained from measuring the fourth emissions to process a second quantification for the blood. Any information/values that fall outside of an anticipated threshold value for the second type of information, are preferably disregarded and not used when determining the second quantification (e.g., an ESR assessment).

In one embodiment, the analyzer-unit (e.g., 51, 81) utilizes a compact user interface display (not shown in detail, but would be on the exterior of housing 51, 81), with separate multi-sensor-element cartridges (e.g., at 54,54′, 154) adapted to determine a quantification, or provide a quantitative assessment such as: {1} determining activated clotting time (ACT) as a function of heparin concentration; {2} simultaneously monitor the blood coagulation profile (TEG) and settling rate (ESR); and {3} determine platelet aggregation by comparison of a thrombin, fibrin, and fibrin+platelet induced clots. The baseline resonance characteristics of the sensor elements 53a-d, 53a′, 83a-d within the cartridge would enable automatic identification by the reader. To perform a measurement, the user first collects a blood sample (with or without additive mixed) with a syringe device 56, 56′, 86a-c, then injects the blood into the cartridge. The user then inserts the cartridge into the sensor detector unit 51, 81, for an automatic quantitative assessment of the blood. Once made, the cartridge sub-unit can be disposed (so as not to cause contamination since it contained a patient's blood).

FIG. 18 is a flow diagram detailing a method 140 for automatically determining a quantification for platelet contribution to clot formation in whole blood or platelet-rich plasma (PRP) using magnetoelastic sensor elements. First, one or more samples comprising the patient's blood is prepared and provided 141: The blood to be analyzed might have traces of a drug being—or recently been—administered to the patient, and/or the sample might be a mixture of the patient's blood and one or more activators/additives, as suggested by box 141. The sample(s) are injected (or otherwise positioned in a manner to minimize contamination of the sample) into a respective sensing bay/chamber integral to a detection cartridge; each bay preferably containing a magnetoelastic element sized/shaped and calibrated for collection of emissions once placed within a time-varying EM field, so as to obtain selected information. The cartridge bays are positioned 144 so as to expose each element to a time-varying magnetic field (such as is created by activating the coils—using techniques detailed by applicants in earlier work—located within a detector unit 51, 81). Emissions from each sensor element in contact with a respective blood sample are measured 146 by the detector unit 51, 81 for processing 147 to provide the quantitative assessment/to quantify one or more property of interest of the patient's blood. For another patient, 148b a new sample is prepared 141 using—preferably—a clean cartridge; if none, 148a, 149, the old cartridge is properly disposed of according to regulations concerning similar bio-hazard substances.

FIGS. 19 and 20 are flow diagrams detailing, in each case, core as well as additional steps of method embodiments, respectively at 90 and 110, for automatically determining a quantification for platelet contribution to clot formation in whole blood or platelet-rich plasma (PRP) using magnetoelastic sensor elements.

Turning, first, to FIG. 19, at least a first and second (and in this particular embodiment, also a third) sample of blood is provided 91 so as to quantify platelet contribution to clot formation within the blood of a mammal. Additional steps 91-96 may comprise: (a) measuring a first resonance amplitude from first emissions collected from a first magnetoelastic sensor element in contact with a first blood sample from the mammal within which a thrombin-activated clot has been generated (to which kaolin, for example, has been added); (b) measuring a second resonance amplitude from second emissions collected from a second magnetoelastic sensor element in contact with a second blood sample from the mammal within which a fibrin clot has been activated (to which a fibrinogen activator such as reptilase/ActivatorF, for example, has been added); and (c) measuring a resonance amplitude of third emissions collected from a third magnetoelastic sensor element in contact with a third blood sample having been activated to result in platelet aggregation (a blood sample to which a platelet activator such as ADP, for example, and a fibrinogen activator such as reptilase/ActivatorF, for example, has been added). If resonance frequency amplitude is not used 96, obtaining selected information about the behavior of each element may be accomplished by employing one or more of the techniques co-developed by applicants hereof, such as by determining Q-factor(s) of the resonance, or otherwise tracking the change in resonance over time of a sensor element's emissions, or any other suitable technique described and referenced in applicants' co-pending parent application Ser. No. 11/710,294 to measure emissions to obtain information about the blood in a sample, including those described elsewhere: (a) the impedance analysis technique applied to measure steady-state vibration of a magnetoelastic sensor element forced by a constant sine wave excitation, and (b) the threshold-crossing counting technique invented and patented earlier by three co-applicants hereof (Drs. K. Zeng, K. G. Ong, and C. A. Grimes).

By measuring changes in the resonance frequency and resonance amplitude of each sensor when in contact with a respective blood sample taken from a mammal/patient (each blood sample having been combined with one or more selected agent), three separate parameters are determined: measurements are taken from the first magnetoelastic sensor element in contact with the first blood sample, regarding behavior of a ‘total activated’ clot; measurements are taken from the second magnetoelastic sensor element in contact with the second blood sample, regarding behavior of the fibrin effect (fibrin clotting cascade) of that mammal's blood; and measurements are taken from the third magnetoelastic sensor element in contact with the third blood sample, regarding the effect due to fibrin and platelets (fibrin and platelet clotting behavior) of that mammal's blood. Information gleaned from measurements taken from the second sensor element about the fibrin effect alone, is subtracted from that gleaned from measurements taken from the third sensor element regarding the combined effect of fibrin and platelets to isolate a collection of diagnostic information about the platelet clotting behavior, alone (box 97).

An associated system system/analyzer-unit includes a detection unit housing a device for generating the time-varying magnetic field(s), the first, second, and third magnetoelastic sensor elements, and a bay/cavity for receiving, respectively, each of the first, second, and third blood samples. As explained elsewhere herein, each sample can be received by ‘injection’ into a respective cavity within which a respective one of the sensor elements is positioned. In operation, an analyzer-unit associated with FIG. 19 performs steps including: measuring first emissions collected from a first magnetoelastic sensor element in contact with a first blood sample from a mammal within which a thrombin activated clot has been generated; measuring second emissions collected from a second magnetoelastic sensor element in contact with a second blood sample from the mammal to which an activator for generating a fibrin clot has been added; measuring third emissions collected from a third magnetoelastic sensor element in contact with a third blood sample from the mammal having been activated to result in platelet aggregation; and processing information from the first, second, and third emissions so collected to make at least one quantitative assessment about the blood.

The characterization of the invention as depicted in FIG. 20 is a method 110 for automatically quantifying, i.e. automatically determining or providing a quantification or quantitative assessment, of one or more selected property (preferably of some diagnostic value). For example 117, information may be about fibrin effect alone, fibrin-platelet interaction, combined effect of activators, platelet clotting behavior, TEG-type reading, ESR-type reading, and so on. At least a first sample (in this case, other samples may or may not be prepared) of blood is provided 111. At least one sensing bay/cavity is at least partially filled with a blood sample 112. The cartridge is positioned 114 so as to expose each sensor element within each bay to a time-varying magnetic field generated by a respective coil housed within a detector unit. Next step 116 is to measure emissions collected from the sensor element in contact with the blood sample (might be the sample within which thrombin-activated clot(s) have been generated, fibrin clots(s) have been activated, activation has resulted in platelet aggregation, and so on), to obtain selected information about resonance frequency behavior of element, a resonance frequency amplitude, track resonance frequency, a Q-factor, and so on.

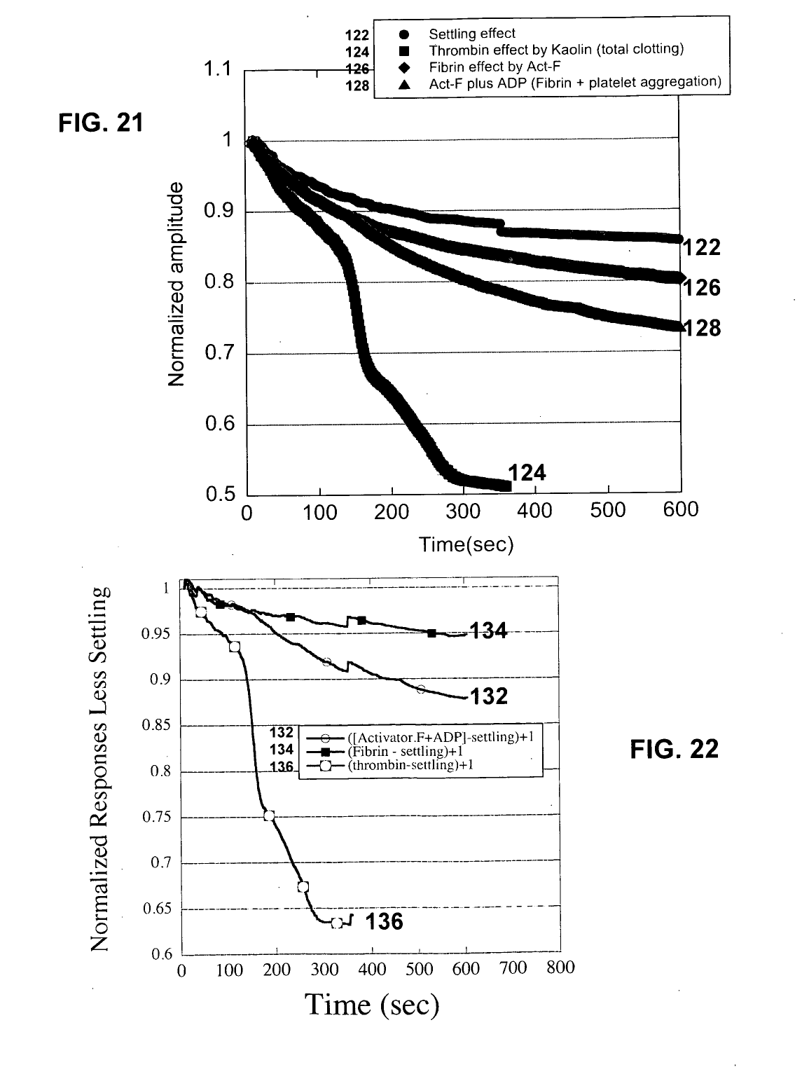

FIG. 21 graphically represents the normalized time dependent change in measured resonance amplitude of a magnetoelastic sensor element immersed in each of four blood sample mixtures. This graphical representation of the method helps one to visualize how isolating information relating to platelet clotting kinetics, from the other information, is done by taking into account effect on the readings with each sensor element associated with particle settling (curve 122 which depicts response over time of settling effect). Curve 124 depicts sensor element response over time when element is immersed in a sample composed of blood and Kaolin (showcases thrombin effect, or ‘total clotting’ situation). Curve 126 depicts sensor element response over time when immersed in a sample composed of blood and Activator-F (showcasing behavior of the fibrin effect/fibrin clotting cascade). Curve 128 depicts sensor element response over time when immersed in a sample composed of blood and Act-F plus ADP (showcasing fibrin and platelet clotting behavior of the patient's blood).

FIG. 22 graphically represents ‘settling-compensated’ (i.e., the settling effect has been subtracted from data) normalized, time dependent change in measured resonance amplitude of magnetoelastic sensors immersed in the blood sample mixtures shown. Curve 132 depicts a ‘normalized’ sensor response over time when element is immersed in a sample composed of blood and Act-F plus ADP (showcasing fibrin and platelet clotting behavior), from which settling effect has been subtracted. Curve 134 depicts ‘normalized’ sensor response over time when element is immersed in a sample composed of blood and Act-F (showcasing behavior of the fibrin effect/fibrin clotting cascade), from which settling effect has been subtracted. Curve 136 depicts a ‘normalized’ sensor response over time when element is immersed in a sample composed of blood and Kaolin (thrombin effect/‘total clotting’), from which settling effect has been subtracted.

As mentioned, quantitative assessment(s)—different types of quantifications—which can be made as contemplated herein, include among others: quantifying platelet aggregation to determine platelet contribution toward clot formation; quantifying fibrin network contribution toward clot formation; quantifying platelet-fibrin clot interactions; quantifying kinetics of thrombin clot generation; and quantifying platelet-fibrin clot strength. The first blood sample comprising a blood product obtained from the mammal selected from the group consisting of: whole blood; and platelet-rich plasma.

Unique structural aspects of an analyzer-unit, FIGS. 12,13,14,15,16A-16B, and 17A-17B include, among others: a cartridge having at least one bay within which a magnetoelastic sensor element is positioned; each bay is in fluid communication with both (a) an entry port for injecting a first blood sample composed of blood taken from a patient (human or other mammal), and (b) a gas vent through which air displaced by injecting the first blood sample into the bay, can be expelled to accommodate the first blood sample. The gas vent comprises a porous plug through which air can be expelled upon injecting the first blood sample. Once air has been expelled through the porous plug, it generally seals against loss of the blood sample. The analyzer-unit is adaptable for testing a sample of blood from a patient to whom a drug is being administered, and therefore likely present in the patient's blood (e.g., an antiplatelet drug discussed, further, below). The analyzer-unit may be comprised of a plurality of bays, all in fluid communication with the same entry port for injecting a first blood sample composed of blood taken from a patient, and (b) a gas vent through which air displaced by injecting the first blood sample into the bay, can be expelled to accommodate the first blood sample. Alternatively, the analyzer-unit may be comprised of a plurality of bays, each bay being in fluid communication with a respective entry port and an associated gas vent through which air displaced by injecting a respective blood sample into the respective bay, can be expelled.

The new system/analyzer-unit and method using magnetoelastic sensor elements as contemplated herein, may also be employed for quantitative assessment of the blood of a patient to which some drug is being administered, for example, an antiplatelet drug (as typically administered, inhibit platelet aggregation and clot retraction). As is known, inhibition of platelet function by administering an antiplatelet drug to a patient permits a care giver to make a general quantitative assessment of the contribution of fibrinogen to clot strength. For instance, one such technique (MTEG)—i.e., a modification of the classic thromboelastograph (TEG) test—uses monoclonal antibody, c7E3 Fab, an antiplatelet drug, was developed by another group, Greilich, et al. (1996). Furthermore, prior use has been made by others in the monitoring of blood coagulation using another modification of traditional thrombelastography (TEG), of an antibody fragment that binds to platelet glycoprotein IIb/IIIa (known as “abciximab-fab”); this antibody fragment abciximab-fab blocks the interaction of platelets with fibrin. The new system/platform/unit and method may also be employed for quantitative assessment of a patient's blood in the event abciximab-fab is being administered to the patient.

A larger sensor tends to provide a stronger signal and better accuracy, but longer sensors are prone to bending that lowers the desired signal amplitude. On the other hand, a sensor element on the smaller size tends to have a weaker signal and lower signal-to-noise ratio. More likely than not, sensor dimension affects the sensitivity because sensors of different dimension have different magnetoelastic properties due to the ΔE-effect, which leads to different stress and mass sensitivities. The dimension of each rectangular-shaped sensor element may be on the order of, say, 10 mm×4 mm. This size is small enough for compact sensor cartridges, but large enough for a strong signal and ease of handling in fabrication. The sensor dimensions can be varied (within the limits of the coil size) according to the sensitivity requirements in a particular measurement with the analyzer-unit generally only requiring a re-calibration in connection with an anticipated new resonance frequency for the sensor element.

Magnetoelastic sensor elements for this example were used on a disposable basis. Total sensor cost is largely determined by the cost of processing, i.e., material handling, the available magnetoelastic ribbon material is quite inexpensive. While sensor elements may be fabricated by a variety of cutting means from a continuous piece of ribbon, mechanical shearing was preferred for this example for its low cost and ease of manufacture. When mechanically sheared, the raw sensor material (ribbon form) can be fed through a metal cutting machine and chopped to a preselected dimension. When a sensor element is mechanically sheared, it contains stresses around the edges that may alter the sensor response in unpredictable ways. Hence, preferably, the magnetoelastic strips are annealed to release these stresses, resetting all sensors to the same magnetic and magnetoelastic states, and also increasing the permeability and magnetoelastic coupling of the sensors. The annealing temperature can be optimized depending on the sensor size, and can also be performed in the presence of a magnetic biasing field to induce an overall magnetic moment in the sensor.

### EXAMPLES

### Analyzer-Unit Having at Least Two Magnetoelastic Elements has Been Tested, Each Element Adapted for Performing a Sensing Function, such as, Quantifying/Characterizing Blood Clot Strength, Quantifying Platelet Aggregation, or Determining Platelet Contribution to Clot Formation in Whole Blood and Platelet-Rich Plasma (PRP)

### FIGS. 21 and 22 Graphical Representations Obtained Using the Following Samples:

Fresh bovine blood from healthy cows was drawn into citrate and heparin tubes (Vacutainer system, BD Biosciences, New Jersey). Activated Clotting Time (ACT) tubes containing 12 mg of kaolin, and ADP tubes containing 20 μM ADP for 1 mL blood were obtained from Helena Laboratories (Texas, USA). Reptilase (Batroxobin™ Maranhao) in the form of 100BU/vial was used Centerchem, Inc. (Connecticut, USA).

To generate a thrombin activated clot, 2 mL citrate anti coagulated blood was injected into an ACT mixed by inversion. 50 μL of 1M CaCl₂ was then pipetted into the blood-kaolin mixture, and the resultant blood sample placed in a glass vial containing a magnetoelastic sensor. The resonance amplitude of the sensor was continuously recorded for ˜10 mins. For activating a fibrin induced clot 50 μL of Batroxobin™ solution (100 BU/vial powder reconstituted in 1 mL of de-ionized water) was added to 1 mL of heparinized blood, and the resulting blood mixture placed in a glass vial containing a magnetoelastic sensor and the resonance amplitude of a sensor immersed in this blood sample recorded. Another sample (targeting the effect of platelet aggregation) was prepared with 1 mL of the Batroxobin™ treated blood added to a tube containing 20 μM ADP and mixed gently. The resonance amplitude of a magnetoelastic sensor immersed in this blood sample was then recorded.

It was observed that blood cells tend to settle onto the magnetoelastic sensor surface affecting resonance amplitude at the beginning of the clotting process. To isolate the effect of settling, i.e., precipitation from the blood onto the gravimetric sensors, the resonance amplitude of a magnetoelastic sensor was measured in a 1 mL citrated blood sample without any additives.

As a result of blood ‘settling’ on the magnetoelastic sensor surface, the amplitude decreases to about 0.85 from the initial value of 1. In case of reptilase (denoted as Activator-F, or Act-F) activated blood, a relatively weak clot formed due to the fibrin network formation and the amplitude decreases to about 0.80. A relatively stronger clot is formed as a result of ADP activation in combination with Activator F (denoted FADP) due to platelet aggregation with the amplitude reducing to 0.73. Finally, when a blood sample is activated with kaolin, the strongest possible clot formed due to thrombin formation and the amplitude saturates at 0.51.

With the saturation amplitude values proportional to the clot strength as measured by a conventional TEG system, platelet aggregation can be estimated using this data. Percentage platelet aggregation is expressed as:

% platelet aggregation=[(MA_{FADP}−MA_{Fibrin})/(MA_{Thrombin}−MA_{Fibrin})]×100 (5)

Where MA represents the normalized saturation measured amplitude value with subscripts indicating the respective activating agents.

Case 1: Settling Not Accounted for in Data Analysis

Using the normalized data without taking into account changes seen in the sensor performance due to settling, platelet aggregation using magnetoelastic sensor amplitude data can be expressed as:

[{MA_{FADP}−MA_{Fibrin}}/{MA_{Thrombin}−MA_{Fibrin}}]×100 (6)

MA_{FADP}=0.73; MA_{Fibrin}=0.8; MA_{Thrombin}=0.51. For which the calculated platelet aggregation is 24.1%.

Case 2: Settling Accounted for in Data Analysis

Compensating the data by the amplitude reduction due to blood settling, platelet aggregation using magnetoelastic sensor amplitude data can be expressed as:

[{(MA_{Settle}−MA_{FADP})−(MA_{Settle}−MA_{Fibrin})}/{(MA_{Settle}−MA_{Thrombin})−(MA_{Settle}−MA_{Fibrin})}]×100 Eqn. (7)

Using the data from FIG. 22: (MA_{Settle}−MA_{FADP})=0.12; (MA_{Settle}−MA_{Fibrin})=0.05; (MA_{Settle}−MA_{Thrombin})=0.37. A platelet aggregation value of 22.1% was obtained for the bovine blood sample used in the present study.

### Conversion of Blood Clot Profile to TEG Data Using New Sensor Elements

Initial experiments to obtain TEG and ESR profiles using an analyzer-unit structured as contemplated herein, were performed on bovine blood injected into the sensor chambers of the cartridge using a 1 mL syringe. The blood for the ESR tests preferably can be citrated to prevent clotting; a suitable amount of calcium chloride (1 M solution in saline) was added to blood samples bound for TEG analysis to nullify the effect of the anticoagulant. Once the cartridge bays were at least partially filled with blood it was placed (with or without syringe attached) inside the coils for detection.

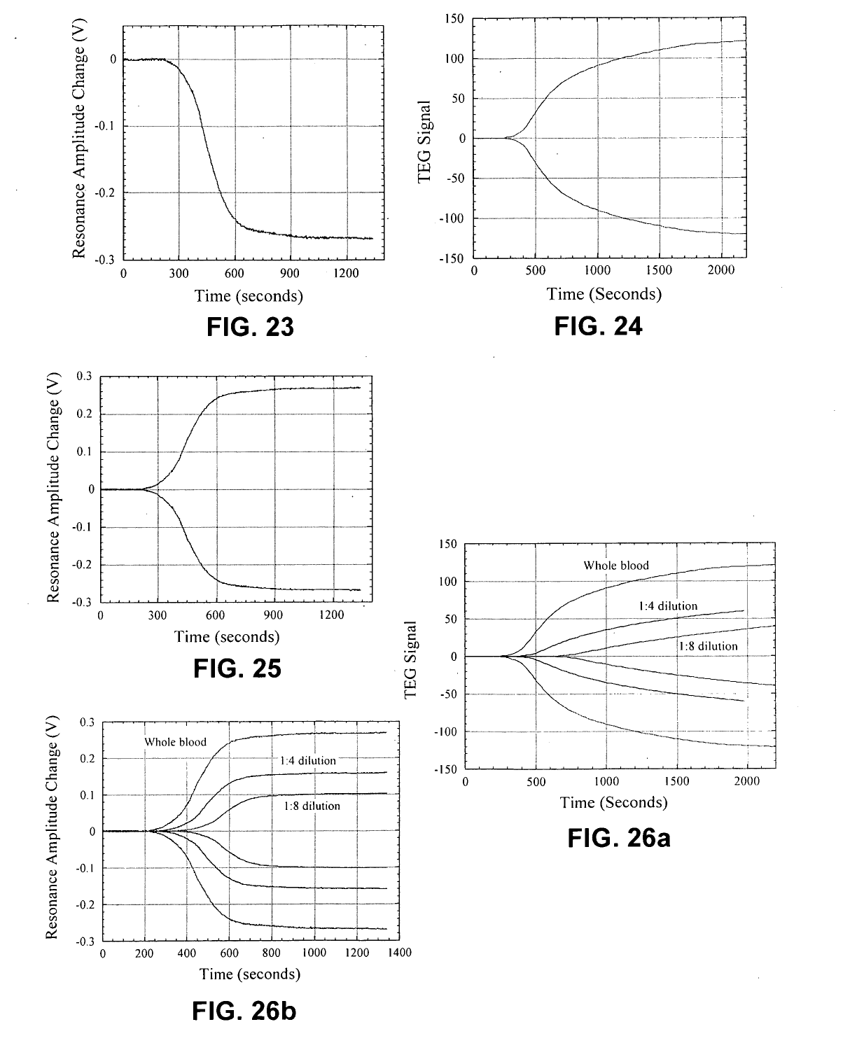

A magnetoelastic sensor element was immersed in a blood sample and both were exposed to a time-varying magnetic field, emissions from which were captured the clot profile of a blood sample by determining the changes in the resonance amplitude of the sensor. FIG. 23 shows the clot profile of a bovine blood sample captured by a magnetoelastic sensor. As shown in the plot, the resonance amplitude of the sensor decreases with blood clot formation. The clot profile is similar to the lower half of the TEG curve shown in FIG. 24. As a result, the curve in FIG. 23 is mirrored (by drawing another line with the same amplitude but opposite sign), and the resulting curve/shape, shown in FIG. 25 is now analogous to FIG. 24. FIGS. 26a,b show the TEG curves for three different blood concentrations (whole blood, 1:4 dilution and 1:8 dilution) measured by: FIG. 26a a Haemoscope TEG® analyzer, and FIG. 26b the magnetoelastic sensor system. From FIGS. 26a,b, one can appreciate: A clot profile generated by a magnetoelastic sensor can therefore be compared with a TEG profile after database compilation.

### Conversion of Settling Rate to ESR

One effective way to correlate settling profiles to ESR values is to perform side-by-side comparisons. This process begins by determining the settling rate S from the measured settling profile (see FIG. 6 hereof) with the equation:

$$S = \frac{{V}_{2} - {V}_{1}}{{t}_{2} - {t}_{1}} \qquad Eqn . ( 8 )$$

where V₂ and V₁ are respectively the sensor signal amplitude at the beginning of the experiment and after a time duration (for example, 10 minutes), and t₂ and t₁ are the times corresponding to V₂ and V₁. A reference data sheet can be constructed with the ESR values of a large number of similar blood samples run on an ESR device. These two data sets can be plotted and a function F defined, such that B=F(A), where A and B represents ESR data points obtained from the magnetoelastic sensor and a commercial device respectively. By obtaining the function F, the actual ESR value B can be determined by substituting the measured S value for any blood sample as A in Eqn. (8).

While certain representative embodiments and details have been shown for the purpose of illustrating features of the invention, those skilled in the art will readily appreciate that various modifications, whether specifically or expressly identified herein, may be made to these representative embodiments without departing from the novel core teachings or scope of this technical disclosure. Accordingly, all such modifications are intended to be included within the scope of the claims. Although the commonly employed preamble phrase “comprising the steps of” may be used herein, or hereafter, in a method claim, the applicants do not intend to invoke 35 U.S.C. §112¶6 in a manner that unduly limits rights to its innovation. Furthermore, in any claim that is filed herewith or hereafter, any means-plus-function clauses used, or later found to be present, are intended to cover at least all structure(s) described herein as performing the recited function and not only structural equivalents but also equivalent structures.

We claim:

1. A method for determining a quantification for blood taken from a patient using information obtained from emissions measured from each of at least a plurality of magnetoelastic sensor elements being exposed to a time-varying magnetic field, the method comprising the steps of:
(a) measuring first emissions collected from a first magnetoelastic sensor element in contact with a first blood sample within which a thrombin-activated clot has been generated;
(b) measuring second emissions collected from a second magnetoelastic sensor element in contact with a second blood sample within which a fibrin clot has been activated;
(c) measuring third emissions collected from a third magnetoelastic sensor element in contact with a third blood sample having been activated to result in platelet aggregation; and
(d) subtracting information obtained from said step of measuring second emissions from information obtained from said step of measuring third emissions to determine the quantification comprising information about platelet clotting behavior of the blood.

2. The method of claim 1:
(a) wherein the patient is selected from the group of animals consisting of humans and non-humans; said information obtained from said step of measuring second emissions comprises information about behavior of the fibrin effect, alone; and said information obtained from said step of measuring third emissions comprises information about behavior of the combined effect of fibrin and platelets; and
(b) further comprising, prior to said steps of measuring first, second, and third emissions, the step of injecting each of said blood samples respectively comprising the blood and a respective first, second, and third additive, into a respective first, second, and third bay containing a respective one of said first, second, and third sensor elements.

3. The method of claim 1 further comprising, prior to said steps of measuring first, second, and third emissions, the steps of:
(a) injecting said first blood sample comprising the blood to which kaolin has been added into a first bay containing said first sensor element;
(b) injecting said second blood sample comprising the blood to which a fibrinogen activator has been added into a second bay containing said second sensor element; and
(c) injecting said third blood sample comprising the blood to which a platelet activator and a fibrinogen activator have been added into a third bay containing said third sensor element.

4. The method of claim 1 wherein:
(a) the blood was taken from the patient while an antiplatelet drug was being administered thereto; and
(b) each of said steps of measuring first, second, and third emissions further comprises measuring a respective first, second, and third, resonance amplitude for each of said respective first, second, and third emissions collected.

5. A method for determining a quantification for blood taken from a patient using information obtained from emissions measured from each of at least a plurality of magnetoelastic sensor elements being exposed to a time-varying magnetic field, the method comprising the steps of:
(a) measuring first emissions collected from a first magnetoelastic sensor element in contact with a first blood sample to obtain first information relating to a first property of the blood;
(b) measuring second emissions collected from a second magnetoelastic sensor element in contact with a second blood sample to obtain second information relating to a second property of the blood, said first information being different from said second information; and
(c) processing said first and second information relating, respectively, to said first and second property of the blood, to determine the quantification.

6. The method of claim 5 wherein:
(a) each of said steps of measuring first and second emissions further comprises measuring a respective first and second resonance amplitude for each said respective first and second emissions collected; and
(b) the quantification for the blood is selected from the group consisting of: quantifying platelet aggregation to determine platelet contribution toward clot formation; quantifying fibrin network contribution toward clot formation; quantifying platelet-fibrin clot interactions; quantifying kinetics of thrombin clot generation; and quantifying platelet-fibrin clot strength.

7. The method of claim 5 wherein each of said steps of measuring first and second emissions further comprises employing a technique selected from the group consisting of: determining a Q-factor of resonance for respective first and second emissions; measuring steady-state vibrations of said respective first and second sensor element where the time-varying magnetic field comprises a constant sine wave excitation; and a threshold-crossing counting technique.

8. A method for determining a quantification for blood taken from a patient using information obtained from emissions measured from each of at least a plurality of magnetoelastic sensor elements being exposed to a time-varying magnetic field, the method comprising the steps of:
(a) measuring first emissions collected from a first magnetoelastic sensor element in contact with a first blood sample;
(b) measuring second emissions collected from a second magnetoelastic sensor element in contact with a second blood sample, said second sensor element and said first sensor element calibrated to provide a first type of information, said first and second blood samples of the same composition;
(c) measuring third emissions collected from a third magnetoelastic sensor element in contact with a third blood sample, said third sensor element calibrated to provide a second type of information; and
(d) comparing information obtained from said step of measuring first emissions with that obtained from said step of measuring second emissions, and processing a first quantification for the blood using said information obtained from measuring said first emissions and said second emissions.

9. The method of claim 8 wherein said step of comparing information further comprises:
(a) disregard any of said information obtained from measuring said first emissions or that obtained from measuring said second emissions, that falls outside an anticipated threshold range; and
(b) in the event both said information obtained from measuring said first emissions and that obtained from measuring said second emissions fall outside said anticipated threshold range, do not process said first quantification, but rather, communicate that an error has occurred.

10. The method of claim 8 wherein:
(a) said first quantification is an average of said information obtained from said measuring first emissions and that obtained from said measuring second emissions, and provides a TEG type assessment for the blood; and
(b) said second type of information provides an ESR type assessment.

11. An analyzer-unit for determining a quantification for blood taken from a patient using information obtained from emissions measured from at least one magnetoelastic sensor element being exposed to a time-varying magnetic field, the analyzer-unit comprising:
(a) integral with a cartridge unit is a bay within which the magnetoelastic sensor element is positioned;
(b) said bay in fluid communication with both (1) an entry port of said cartridge unit for receiving a blood sample comprising the blood taken from the patient, and (2) a gas vent generally permeable to air and generally impermeable to said blood sample; and
(c) a detector sub-unit housing at least one coil for generating the time-varying magnetic field, an interior space of said coil having a cavity sized for receiving said bay of said cartridge unit.

12. The analyzer-unit of claim 11, further comprising:
(a) integral with said cartridge unit is a second bay within which a second magnetoelastic sensor element is positioned;
(b) said second bay in fluid communication with both (1) said entry port for receiving, by injection, said blood sample, and (2) a second gas vent generally permeable to air and generally impermeable to said blood sample; and
(c) said detector sub-unit further housing a second coil, an interior space of which has a cavity sized for receiving said second bay of said cartridge unit.

13. The analyzer-unit of claim 12, further comprising:
(a) integral with said cartridge unit is a third bay within which a third magnetoelastic sensor element is positioned;
(b) said third bay in fluid communication with both (1) said entry port for receiving, by injection, said blood sample, and (2) a third gas vent generally permeable to air and generally impermeable to said blood sample; and
(c) said detector sub-unit further housing a third coil, an interior space of which has a cavity sized for receiving said third bay of said cartridge unit.

14. The analyzer-unit of claim 13 wherein:
(a) said first sensor element and said second sensor element calibrated to provide a first type of information obtained from measuring, respectively, first emissions collected from said first sensor element in contact with said blood sample and second emissions collected from said second sensor element in contact with said blood sample; and
(b) said third sensor element calibrated to provide a second type of information obtained from measuring third emissions collected from said third sensor element in contact with the blood sample.

15. The analyzer-unit of claim 11, further comprising:
(a) integral with said cartridge unit is a second bay within which a second magnetoelastic sensor element is positioned;
(b) said second bay in fluid communication with both (1) a second entry port for receiving a second blood sample, and (2) a second gas vent generally permeable to air and generally impermeable to said second blood sample; and
(c) said detector sub-unit further housing a second coil, an interior space of which has a cavity sized for receiving said second bay of said cartridge unit.

16. The analyzer-unit of claim 15 wherein:
(a) each said blood sample injected into one of said bays, respectively, comprises the blood and a respective one of a first and second additive; and
(b) each said gas vent comprises a porous plug in communication with an exit port through which air is expelled from within said bay upon injecting a respective one of said blood samples therein.

17. The analyzer-unit of claim 16 wherein:
(a) said first additive is a fibrinogen activator so as to activate a fibrin clot within said first blood sample, and said second additive comprises a platelet activator {such as ADP} and said fibrinogen activator so as to result in information regarding fibrin and platelet allotting behavior within said second blood sample; and
(b) subtracting information obtained from said step of measuring first emissions from information obtained from said step of measuring second emissions to determine the quantification comprising information about platelet clotting behavior of the blood.

18. The analyzer-unit of claim 11 wherein:
(a) said entry port is adapted for accepting an end of a syringe within which said blood sample is stored prior to injecting into said bay; and
(b) said gas vent comprises an encased porous plug in communication with an exit port through which air is expelled from within said bay upon injecting said blood sample therein.

19. The analyzer-unit of claim 17 wherein:
(a) each said blood sample injected into said bay comprises the blood and a first additive; and
(b) once said blood sample is injected into said bay, said needle is removed from said entry port which becomes generally impermeable to air and said blood sample so as to close-off said entry port.

20. The analyzer-unit of claim 11 in electrical communication with a processing unit for determining the quantification from information obtained from emissions measured from at the magnetoelastic sensor element while being exposed to a time-varying magnetic field.
